(19) United States
(12) Patent Application Publication
(10) Pub. No.: US 2002/0192194 A1
(43) Pub. Date: Dec. 19, 2002

(54) Production and use of dopaminergic cells to treat dopaminergic deficiencies

(76) Inventors: Michael McGrogan, San Carlos, CA (US); Gary Snable, Atherton, CA (US)

Correspondence Address: John W. Ryan, WILMER CUTLER & PICKERING, 2445 M. Street, N.W., Washington, DC, 20037-1420, US

(21) Appl. No.: 10/000,452

(22) Filed: Dec. 4, 2001

Related U.S. Application Data
(60) Provisional application No. 60/251,735, filed on Dec. 5, 2000.

(51) Int. Cl. A61K 48/00; C12N 5/08

(52) U.S. Cl. 424/93.21; 424/93.7; 435/368

(57) ABSTRACT

Differentiated neuronal cells suitable for transplantation in individuals with a dopamine deficiency are derived from progenitor cells. The progenitor cells are treated with at least one inducing agent such as retinoic acid for a time period sufficient to optimize expression of tyrosine hydroxylase. The cells intended for transplantation are optionally treated with a lithium salt to enhance bcl-2 production and survival. Optionally, the progenitor cells are co-cultured with Sertoli cells, bone marrow stem cells, or a combination thereof. The transplantation-ready cells are isolated and harvested. The resulting neuronal cells are purified and have a phenotype optimized to treat a dopaminergic deficiency, such as Parkinson's Disease. Optionally the neuronal cells can be implanted with Sertoli cells, bone marrow stem cells or a combination thereof.

A purified human dopaminergic cell type is obtained by culturing NT2 cells and treated for about three weeks with an inducing agent, culturing for about two weeks with growth media without an inducing agent or mitotic inhibitor, culturing for about one week with at least one mitotic inhibitor, harvesting and placing in a diluent.

### TECHNICAL FIELD

The present invention is in the field of tissue engineering/transplantation and more particularly dopaminergic cells, their production and use in transplantation.

### BACKGROUND ART

Dopaminergic neurons are those which synthesize and use dopamine (DA) as a neurotransmitter. Dopaminergic neurons are found in a number of areas of the brain, including the nigrostriatal, mesolimbic, mesocortical and tubero-hypophysial systems. The rate-limiting step in dopamine synthesis is catalysis of tyrosine by tyrosine hydroxylase (TH). Dopamine is stored in synaptic vesicles. Dopamine is released from presynaptic vesicles by exocytosis. Dopamine acts on as many as five classes of receptors. Dopamine is recycled by reuptake and/or degradation by monoamine oxidase B (MAO-B) (RK Murray, Ch. 64. The Biochemical Basis of Some Neuropsychiatric Disorders. In: Harper's Biochemistry, ed. by Murray, et al. 24th ed., Appleton & Lange, Stamford, Conn., 1996, pp. 794-814).

Parkinson's disease (PD) is a neurodegenerative disorder characterized by a loss of dopaminergic cells from the substantia nigra par compacta, resulting in decreased dopaminergic input to the striatum. The hallmark motor symptoms include tremor, rigidity, bradykinesia, and instability. In spite of a host of approved pharmacological and surgical treatments, existing therapies for PD are only partial and palliative. Levodopa (L-dopa) the gold standard pharmacological treatment to restore DA, is plagued by decreased efficacy and increased side effects over time. Adjunct treatment with DA agonists is frequently necessary; however, recently approved DA agonists with greater receptor subtype specificity may provide only incremental clinical benefit. Catechol-O-methyltransferase (COMT) inhibitors to slow DA metabolism soon will be joining monoamine oxidase (MAO) inhibitors.

To replace the missing cells, there has been a renaissance of neurosurgical treatments for PD. After all pharmacological treatments have failed, surgical procedures including pallidotomy, thalamotomy and deep electrical stimulation may be considered. Nevertheless, for almost one million individuals in the US afflicted by PD, a reliable long-term treatment to halt disease progression remains elusive.

Schizophrenia is often treated by neuroleptic drugs which decrease the amount of dopamine activity in mesolimbic dopaminergic neurons. “Positive symptoms” (e.g., hallucinations, delusions, bizarre behavior) have been associated with excess dopamine activity in the mesolimbic neurons. “Negative symptoms” of schizophrenia (e.g., social withdrawal, emotional blunting, and catatonia) may be associated with low dopamine activity in the prefrontal cortex. Since prefrontal dopaminergic neurons may normally inhibit the activity of subcortical dopamine neurons, a lowering of dopamine in the prefrontal area could lead to the elevated dopaminergic activity in the subcortical neurons.

Progressive Supranuclear Palsy (Steele-Richardson-Olszewski Syndrome) is due to a loss of neurons and gliosis in the tectum and tegmentum of the midbrain, the subthalamic nuclei of Luys, the vestibular nuclei, and to some extent the ocular nuclei. Some symptoms are shared with Parkinson's disease, including rigidity of the neck and other trunk muscles and occasional sensitivity to L-dopa.

A rare form of torsion dystonia is dramatically L-dopa-responsive. Starting in childhood, the dystonia first affects gait. Most individuals later develop parkinsonism. Some focal dystonias also are reported to be L-dopa responsive.

In a neurodegenerative disorder associated with autonomic failure (i.e., Shy-Drager Syndrome), positron emission tomography has shown decreased uptake of dopamine derivatives in the putamen and caudate, probably reflecting a loss of nigrostriatal dopaminergic neurons. Current treatment is symptomatic. The parkinsonian symptoms may be helped by L-dopa or other dopaminergic drugs, but later most patients become refractory to these drugs.

Depression is associated with heterogeneous dysregulations of the biogenic amines. Although norepinephrine and serotonin have been most implicated in the pathophysiology, dopamine also may play a role in depression. Dopamine may be reduced in depression and increased in mania. Drugs that reduce dopamine concentrations (e.g., reserpine) and diseases that reduce dopamine concentrations (e.g., Parkinson's disease) are associated with depressive symptoms. Also, drugs that increase dopamine concentrations (e.g., tyrosine, amphetamine and bupropion) reduce the symptoms of depression. Two recent theories regarding dopamine and depression are that the mesolimbic dopamine pathway may be dysfunction in depression and that the dopamine type 1 (D1) receptor may be hypoactive in depression (Ch.9. Mood Disorders, in: CONCISE TEXTBOOK OF CLINICAL PSYCHIATRY. Ed. by H I Kaplan and B J Sadock. Williams & Wilkins, Baltimore, Md., 1996, pp. 159-188).

MAO inhibitors also are the drugs of choice in agoraphobia (irrational fear of being alone or in public places) and panic disorder. There also is growing evidence that MAO inhibitors are effective in the treatment of some anxiety disorders, particularly mixed depressed and anxious states.

The search for a continuous, stable, regulated, site-specific source of DA delivery has turned to tissue transplantation, cell therapy and genetic engineering, with the ultimate goal of finding an effective treatment to halt or reverse disease progression.

Human fetal mesencephalic tissue transplants have been extensively studied. They have demonstrated therapeutic potential in animal models of PD and in Parkinson's disease patients. Fetal tissue transplants have been performed in the clinic for over a decade on more than 200 patients throughout the world with positive outcomes (Kordower J H, Goetz C G, Freeman T B, Olanow C W. Experimental Neurology 144:41-46, 1997). Grafts survive, form synaptic connections, and improve motor function in many patients. However, ethical, moral and technical constraints limit the widespread use of human fetal tissue. Future progress in the field of neural transplantation will depend largely on the development of alternative sources of cells.

Xenotransplantation, the use of cells from different species, is a viable approach to circumventing the limitations associated with human fetal neural transplantation (Galpern W R, Burns L H, Deacon T W, Dinsmore J, Isacson O. Experimental Neurology 140:1-13, 1996). A phase I clinical trial sponsored by Diacrin, Inc., is evaluating transplants of porcine cells harvested from the midbrains of pig fetuses. Another technique, developed by Cytotherapeutics, Inc., uses encapsulated xenografts of rat PC12 cells that secrete dopamine. A semipermeable polymer membrane allows diffusion of the small therapeutic molecules but prevents diffusion of the larger immunogenic molecules. Whether the release of dopamine from encapsulated sources will be sufficient to restore optimal DA levels in PD patients remains to be determined. Although cells derived from animals are potential candidates for human neural transplantation, they carry the risks of transferring intrinsic pathogens, creating novel infectious agents, or eliciting deleterious immune responses (Isacson O, Breakefield X. Nature Medicine 3:964-969, 1997).

Cell therapy for PD, which is still at the experimental stage, is theoretically capable of reversing neurotransmitter deficiencies, halting neural degeneration, and repairing neural damage. Many types of cells (e.g., rat fibroblasts) have successfully been transfected ex vivo with, for example, the human tyrosine hydroxylase (TH) gene to serve as a local source of dopaminergic factors (Raymon H K, Thode S, Gage F H. Experimental Neurology 144:82-91, 1997). Concerns about long-term stable gene expression, tumor formation, and pathogen delivery need to be resolved.

In vivo gene therapy is possible using direct insertion of genes into brain cells via viral vectors (herpes simplex virus, adenovirus, adeno-associated virus, or lentivirus). Vectors encoding genes such as TH or glial-derived neurotrophic factor have been genetically engineered into cells lines. However, transplantation of genetically engineered cells into animal models of PD has not provided conclusive long-term beneficial effects or reinnervated the dopamine depleted striatum. Moreover, the extent of gene expression, long-term efficacy, and cytopathogenicity associated with viral vectors is unknown.

Growth factors such as GDNF and brain-derived neurotrophic factor (BDNF) can be delivered alone or in combination with tissue transplants to provide trophic support and protect dopaminergic cells (Rosenblad C, Matinez-Serrano, Bjorklund A. Neuroscience 75:979-985, 1996). The long-term benefits and risks are unknown. Delivery is problematic, but novel approaches via injection directly into the brain, a Medtronic device, encapsulated cells, and genetically engineered cells are under investigation.

Recent research has focused on adapting NT2 or hNT cells for treatment of Parkinson's Disease (Iacovitti and Stull, NeuroReport 8:1471-74, 1997). Both newly differentiating human neurons (hNT cells) and the undifferentiated precursors (NT2 cells) were treated with a variety of factors. In hNT neuronal cells but not NT2 precursor cells, TH expression was only induced by a combination of aFGF and co-activators (DA, TPA, or IBMX/forskolin), not individual factors. With increasing time in culture, more hNT cells expressed TH. After five days, 565 out of 10⁵ plated hNT cells, or less than 1%, expressed TH.

Lithium, the primary treatment for mania and bipolar affective disorder, has been reported to significantly influence the activity of signaling systems. Using PC12 cells as a model system, Li and Jope (J Neurochem 65:2500-08, 1995) studied the NGF-induced expression of several signal transduction proteins, including subtypes of G proteins, protein kinase C and phospholipase C and its modulation by lithium. Their results demonstrated that lithium, at a therapeutic concentration (1 mM), modulates the level of signal transduction proteins. Several studies have indicated that the activation of TH by intracellular calcium ion could be mediated by calcium/calmodulin-dependent protein kinase (for review, see Masserano et al., “The Role of TH in the Regulation of Catecholamine Synthesis.” In handbook of experimental pharmacology. Vol 90/II Catecholamines, Ed. by Trendelenburg and Weiner, Springer Verlag, Berlin, 1990, pp 427-69). However, controversial results have been obtained when lithium has been studied in relation to the brain content of catecholamines. Both decreased synthesis of dopamine (Friedman and Gershon, Nature 243:520-21, 1973) and up-regulated TH activity (Segal et al., Nature 254:58-59, 1975) have been reported after lithium treatment, perhaps due to the complexity of the brain tissue. On the other hand, increased synthesis and secretion of catecholamines and protein kinase C activity was demonstrated (Terao et al., Biol Psychiatry 31:1038-49, 1992) when lithium was applied on cultured adrenal medullary cells.

In summary, there is substantial evidence in both animal models and human patients that neural transplantation is a scientifically feasible and clinically promising approach to the treatment of PD. Nevertheless, alternative cell sources and novel strategies are needed to circumvent the numerous ethical and technical constraints that now limit the widespread use of neural transplantation.

According to Anton et al. (Anton R, et al. Exp Neurol 127:207-218, 1994), the ideal cell for a CNS transplant system should meet the following criteria: It should be of human CNS origin, capable of growth cessation and differentiation, clonal and defined, transfectable and selectable, immunologically inert, capable of long-term survival following implantation, non-tumorigenic, functional and integrated into the host brain, of consistent quality, and readily available and hNT Neurons meet all these criteria.

### BRIEF DESCRIPTION OF DRAWINGS

FIG. 1 is a tyrosine hydroxylase Western blot of NT cells at different stages of maturation in the hNT neuron process. It was developed with anti-TH monoclonal antibody and biotin-streptavidin alkaline phosphatase system. The following lanes contain: 1) 500 pg TH rat standard, 2) 1×10⁶ purified hNT neurons, as positive control, 3) 1×10⁶ Replate-I Neurons, 4) 1×10⁶ Replate-I Accessory cells, 5) 1×10⁶ NT2/D1 Precursor cells. Lanes 6-8 contain 1×10⁶ Replate-II neurons each harvested after 1 week (lane 6), 2 weeks (lane 7), and 3 weeks (lane 8) in extended culture.

FIG. 2 is a tyrosine hydroxylase Western blot comparing different maturation conditions for the hNT neurons. It was developed with anti-TH monoclonal antibody and biotin-streptavidin alkaline phosphatase system. NT2/D 1 cells were induced with RetA for 6 weeks and processed as Replate-I or Replate-II cultures in mitotic inhibitors for 1 week. Then the cultures were allowed to mature in conditioned media for 1 day (1 week replate), 1 week (2 week replate), or 2 weeks (3 weeks replate). Pure hNT neurons were harvested from the mature replate cultures and cell extracts corresponding to 1×10⁶ cells were loaded in the following lanes. Lanes 1-3 show the results for the Extended Replate-I Neurons which were matured for 1 week (lane 1), 2 weeks (lane 2), and 3 weeks (lane 3). Lanes 5 and 6 have Replate-II Neurons which were matured for 1 week and 2 weeks, respectively. Lanes 4 and 7 contain 1×10⁶ hNT neurons positive control.

FIG. 3 is a TH Western Blot showing the time course of RetA Induction. It was developed with anti-TH monoclonal antibody and biotin-streptavidin alkaline phosphatase system. The NT2/D1 cells were induced with RetA for 4, 5, or 6 weeks, and after induction Replate-I cultures were maintained in mitotic inhibitors for either 1 week (Lanes 2-4) or a total of 2 weeks matured (Lanes 5-7). Purified neurons were harvested from each sample and cell extracts corresponding to 1×10⁶ cells were loaded in the following lanes: for the 1 week Replate-I: 2) 4w-RetA, 3) 5w-RetA, 4) 6w-RetA; and for the 2 weeks matured Replate I: 5) 4w-RetA, 6) 5w-RetA, 7) 6w-RetA, 8) hNT positive control; and rat TH standard was in lanes 1 (500pg) and 9 (5ng).

FIGS. 4A-4G are photomicrographs of cultured neurons. DA neurons (4A and 4B) were immunostained for TH (arrows). For FIGS. 4A and 4B, the bar is 50 μm. FIG. 4C is a fluorescent photomicrograph showing TH+hNT neurons (bar is 15 μm). FIGS. 4D and 4E are representative light photomicrographs of DAT-labeled DA neurons cultured an additional day and 5 days, respectively (bar is 50 μm). FIG. 4F shows hNT cells immunolabeled for DAT (bar is 25 μm). FIG. 4G is a fluorescent photomicrograph showing a clump of DA neurons (white asterisks) labeled with TH (green) and DAT (red) (bar is 10 μm).

FIG. 5A and 5B are fluorescent photomicrographs of DA neurons cultured an additional 5 days and labeled for D2 (FIG. 5A, red fluorescence, bar is 50 μm) and for TH (green) and D2 (orange-red) FIG. 5B, bar is 25 μm).

FIGS. 6A-6E are photomicrographs of DA(4 week) and hNT ( 5 week) neurons labeled for AHD-2 and TH. FIGS. 6A shows clumps of DA (4 week) cells labeled for AHD-2, while FIG. 6B show clumps of hNT (5 week) cells similarly labeled. For FIGS. 6A and 6B bar is 50 μm. FIG. 6C shows DA neurons labeled for ADH-2; FIG. 6D shows DA neurons labeled for TH; and FIG. 6E shows double labeling (arrows) for TH and AHD-2 in DA neurons. For FIGS. 6C-6E the bar is 25 μm.

FIG. 7 is a bar graph showing the effects of different doses of lithium chloride on TH expression in cultured hNT neurons (induced for 5 weeks with retinoic acid).

FIGS. 8A through 8C are photomicrographs illustrating the effects of 4 weeks of RetA and 5 days of LiCl on DA neuronal cells on frequency of TH-expressing cells (8A), TH and PI staining (8B) and bcl-2 expression.

FIG. 9 is a bar graph showing the effects of different doses of lithium chloride on Bcl-2 expression in cultured DA neurons.

FIGS. 10A-10E are photomicrographs of representative control and lithium-treated hNT neurons cultured for 5 days and immunostained for tyrosine hydroxylase. FIG. 10A shows a control culture of hNT neurons and reveals few TH-positive cells. FIGS. 10B and 10C show hNT cells cultured with 1.0 mM (10B) and 3.0 mM (10C) lithium chloride. FIGS. 10D and 10E show the representative morphology of TH-positive hNT cells treated with 1.0 mM (10D) and 3.0 mM (10E) of lithium chloride.

FIG. 11 is a table that shows the effect of lithium chloride on soma size of hNT neurons cultured for 5 days.

FIG. 12 is a table that shows the effect of lithium chloride on neurite growth of hNT neurons cultured for 5 days. In FIGS. 11 and 12, the * denotes significant difference (p<0.01) compared to control.

FIGS. 13A-13E are photomicrographs of the distribution, morphological appearance and phenotype of hNT neurons after 5 days in control culture and lithium-treated cultures. FIG. 13A is a phase contrast low-magnification photomicrograph showing the distribution of hNT neurons in control/untreated cultures. FIG. 13B is a higher magnification photomicrograph demonstrating that virtually all cultured hNT cells are immunoreactive for GAP43. FIGS. 13C and 13D are low-magnification phase contrast photomicrographs of hNT neurons treated with 1.0 mM (13C) and 3.0 mM (13D) concentration of lithium chloride. FIG. 13E illustrates the morphological appearance of GAP-43-labeled hNT neurons treated with 3.0 mM lithium chloride.

FIG. 14 compares the TH levels of DA (4 week) neurons maturing under different conditions. Lane 1 contains 500 pg TH, lane 2 is fresh DA-Neurons bulk harvested. After one week of co-culturing DA (4 week) neurons, lane 3 shows the results with a) polylysine +laminin, b) TM-4 cells are in lane 4, and c) rat glial cells are in lane 5.

FIG. 15 shows a Western blot for DA neurons cultured for one or two weeks on polylysine +laminin, TM4 Sertoli cells, SF-126 cells and BMSC (only two weeks).

FIG. 16 is a bar graph comparing the effects of hNT cells, DA neurons, and LiCl-treated neurons on the lesioned animals' performance of rotations in the PD rat model.

FIG. 17 is a bar graph comparing the mean numbers of surviving hNT, DA, and LiCl-treated neurons at the two implant locations (striatum and substantia nigra).

FIG. 18 is a graphical representation of the various time courses of RetA induction, DF media chase incubation and replating.

FIG. 19 shows a Western blot for TH levels of cells cultured in various ways. MI cells (lanes 3 and 7) have higher levels. Lane 1 has 500 pg of TH. Lanes 2, 3 and 4 contained freshly harvested hNT, MI and DA neurons, respectively. Lane 5 contains DA neurons treated during replate with media containing 1 mM LiCl. In the same order, lanes 6-9 contain the same cell types that were tested after freezing and thawing.

FIGS. 20A-20D are photomicrographs of FDA-PI stained cells. FIGS. 20A and 20B show NT2 cells at 1 and 5 DIV. FIGS. 20C and 20D show DA cells and 1 and 5 DIV.

FIGS. 21A-21E are photomicrographs of tdt-labeled cells (apoptotic cells). FIGS. 21A shows apoptotic nuclei in cultured NT2 cells. FIG. 21B shows a group of DA neurons with apoptotic nuclei. FIG. 21C shows a clump of DA neurons with single or multiple lobes of condensed chromatin. FIG. 21D shows MI apoptotic cells. FIG. 21E shows some dying MI neurons. FIG. 21F shows the positive control (treated with nuclease to generate DNA breaks in cells and staining in all cells).

FIGS. 22A-22F are photomicrographs which display the results of TH/DAPI immunocytochemistry. FIGS. 22A-C show TH+ cells, Hoechst 33258 staining, and double-labeling of MI neurons cultured for 1DIV. FIGS. 22D-F show hNT cells. FIG. 22D shows a TH+neuron clump with fragmented nucleus, magnified in FIGS. 22E and 22F.

### DETAILED DESCRIPTION

Dopaminergic neuronal cells suitable for transplantation in dopaminergic deficiency are derived from progenitor cells as follows. The progenitor cells are treated with retinoic acid for a time period sufficient to optimize expression of tyrosine hydroxylase. The optimized neuronal cells are further treated with at least one lithium salt or a combination thereof. The DA neuronal cells are harvested. The resulting neuronal cells are highly purified and have a phenotype optimized for at least one neurodegenerative disease, such as Parkinson's Disease. Optionally, the neuronal cells also are cultured or administered with Sertoli, bone marrow stem, or fetal stem cells.

The six criteria for transplantable cells summarized above are met by DA and MI Neurons. In addition, DA and MI neurons surprisingly were able to be optimized for stable TH production similar to that seen with primary mesencephalic cells. TH is vital because it performs the rate-limiting step in production of dopamine. These optimized DA neuron cells have improved dopaminergic properties arising from manipulating the hNT neuron's natural capabilities. These procedures eliminated the need to transfect the cells with exogenous gene constructs.

DA and MI neurons have the potential to overcome many of the limitations associated with human fetal tissue transplantation, including poor graft survival (5-10%), high tissue variability, and low degree of host re-innervation. hNT neurons have demonstrated excellent graft survival and behavioral improvements in animal models of CNS disorders. There are preliminary data suggesting that hNT neurons may have immunosuppressive properties and produce neuroprotective, neurotrophic factors. Thus, long-term, systemic immunosuppression may not be necessary in humans.

Furthermore, hNT neurons are human cells derived from the human teratocarcinoma NT2/D1 cell line through induction with RetA treatment (Andrews, P W, Damjanov J, Simon D, Banting G, Carlin C, Dracopoli N C, Fogh J. Lab Invest 50:147-162, 1984). During the 6-week retinoic acid induction period, NT2/D1 cells, which share many characteristics of neuroepithelial precursor cells, undergo significant changes resulting in the loss of neuroepithelial markers and the appearance of neuronal markers (Pleasure S J, Page C, Lee V M. J Neurosci 12:1802-1815, 1992; Lee V M, McGrogan M, Lernhardt W, Huvar A. Strategies in Molecular Biol 7:28-31, 1994). Several enrichment steps result in the production of >99% pure populations of hNT neurons that are terminally differentiated (Andrews et al, ibid.). They display process outgrowth and establish functional synapses. Thus, mature hNT neurons do not divide, they maintain a neuronal phenotype, and they appear to be virtually indistinguishable from terminally differentiated post-mitotic, embryonic neurons (Pleasure S J, LEE V M J Neurosci Res 35:585602, 1993).

Finally, we demonstrate in the studies disclosed herein that the phenotype of the NT2/D1 cells can be altered by certain culture conditions—without transfection of foreign genes—to consistently produce the levels of dopamine to ameliorate abnormal dopaminergic conditions.

Definitions:

A dopaminergic deficiency is a condition in which there is a shortage of dopamine. The dopaminergic deficiency may have a variety of causes, including, but not limited to, under-production by dopaminergic neurons, deficit of dopaminergic neurons, or insensitivity of dopaminergic neurons to dopamine. Examples of such conditions include, but are not limited to, Parkinson's disease, schizophrenia, progressive supranuclear palsy (Steele-Richardson-Olszewski Syndrome), and a Dopa-responsive form of torsion dystonia.

“Beneficial effect” is an observable improvement over the baseline clinically observable signs and symptoms. For example, a beneficial effect can include improvements in graft survival, improvements in one or more of the signs and symptoms associated with a dopaminergic deficiency, such as movement or mood.

“Mammal” includes humans and other mammals that would reasonably benefit from treatment, including pets such as dogs and cats.

“NT2/D1 precursor cells” as used herein refers to a special cell line available from Layton Bioscience, Inc. (Atherton, Calif. ). This cell line has been developed from a previously described human teratocarcinoma cell line (termed Ntera2/clone DI or NT2 cells) (Andrews et al. Lab. Invest. 50:147-162, 1981). These cells are precursors for “LBS-Neurons” human neuronal cells. NT2/D1 cells are unique among other teratocarcinoma cell lines because these cells act like progenitor cells whose progeny are restricted to the neuronal lineage (Andrews, ibid.)

“LBS-Neurons” human neuronal cells as used herein refers to the special neuronal cell line disclosed in U.S. Pat. No. 5,175,103 to Lee et al. Briefly, NT2/D1 precursor cells are induced to differentiate into neurons by administration of 10 μM RetA which is replenished twice weekly for 6 weeks, after which the cells are replated with special manipulations to become more than 99% pure hNT neurons. These are the cells that are used in the subsequent experiments. Alternately, for human use, there is a cell line manufactured without antibiotics (used in the research grade hNT-Neurons) and under good manufacturing practices (GMP), which is termed LBS NEURONS human neuronal cells (Layton Bioscience, Inc.).

“Dopaminergic neurons” have a dopaminergic phenotype, including expressing TH, AHD2, DARPP-32 and D2 dopamine receptor. Dopaminergic neurons are obtained by retinoic acid induction of NT2/D1 cells for at least two to three weeks to about 4 weeks. If the NT2 cells are induced with retinoic acid for 4 weeks and then replated with mitotic inhibitors, the resulting neurons are called DA neurons. If the NT2/D1 neurons are induced by retinoic acid for 3 weeks, they also are further matured in DF-media for two weeks and then treated with mitotic inhibitors. This 3W RetA / 2W DF produces “minimally induced” or MI Neurons.

“Inducing agent” includes, but is not limited to compounds that have the same effect in causing NT2/D1 precursor cells to differentiate into hNT neurons, one example of which is retinoic acid. Thus, an inducing agent includes not only retinoic acid in any of it isomers and trans/cis forms, but also similarly active compounds.

“Immunosuppressant” as used herein is a substance which prevents or attenuates immunologic phenomena. For example, such immunologic phenomena include inflammation, autoimmunity, GVHD and graft rejection. Examples of current immunosuppressants include but are not limited to cyclosporine A, cyclophosphamide, prednisone and tacrolimus (FK506). Optionally, an immunosuppressant can be administered at the time of the transplant. One regimen calls for administering the immunosuppressant for two days, before and on the day of transplantation.

“Vehicle” is a biologically compatible solution, such as phosphate buffered saline and the like, which is used to suspend and inject the dopaminergic cells into mammals. Optionally, magnesium and calcium can be added to the vehicle. This definition also includes any gel or matrix which firms at body temperature and is biodegradable.

As used herein, the term “sample” is meant to refer to one or more treated cells. In preferred embodiments, a sample contains a plurality of cells. According to the present invention, a sample of treated cells is implanted into either a non-human mammal or a human.

By “lithium” is meant generally a lithium salt, wherein the anion includes but is not limited to chloride, bromide, carbonate, citrate, or other biologically compatible monovalent anion. In particular, lithium chloride (LiCI) has been used in many of the examples disclosed below.

“Therapeutic agent” as used herein means the transplanted cells themselves or chemical entities secreted by these cells. Examples of chemical entities secreted by the cells include, but are not limited to dopamine, other neurotransmitters, neurotrophic factors, proteins and hormones.

The production of hNT Neurons is an 8-10 week process. All cell culture work has been performed in T-flasks but can be performed in other containers. hNT neurons are induced from NT2/D1 cells following exposure to growth media containing 10 μM RetA for about 5-6 weeks. Cells are harvested using trypsin and replated at reduced density. Replate I cultures are maintained in growth media for 2 days and then separated from the accessory cells by gentle selective harvest to give an enriched neuron population. Extended replate I cultures are plated in media and at 24 hours treated with mitotic inhibitors for 5-10 days. Purified neurons are then selectively harvested using trypsin and formulated for cryopreservation. For some experiments (see examples), the cells were maintained in neuron-conditioned media and allowed to mature in culture. After Ret A induction, hNT neurons constitute approximately 10-20% of the cell population; the remainder are non-neuronal accessory cells. hNT neurons are post-mitotic and no longer capable of dividing; whereas, the accessory cells are mitotically inhibited by the addition of cytosine arabinoside (Ara-C) and fluorodeoxyuridine (FUdR) to culture medium. Harvest results in a purified bulk product of >95% neurons which is formulated in freezing media and cryopreserved.

Initial experiments manipulated hNT cells (induced with RetA for 5 or 6 weeks). Later examples characterized DA neurons (induced with RetA for 3-4 weeks) and subjected to an extended Replate I cycle only.

### EXAMPLES

### Example 1

### RT-PCR for TH and D₁ and D₂ Receptors

Reverse transcript PCR (RT-PCR) was used to detect the presence of endogenous transcribed mRNA for enzymes such as TH. Messenger RNA and cDNA were prepared using commercially available reagents and kits. PCR primers, if not already at hand, were designed using the MacVector software package to minimize primer self annealing and PCR artifacts. PCR was performed using standard parameters (Innis Mass., Gelfand D H, Sninsky J J, White T J. PCR Protocols. Academic Press, 1990) with commercially available enzymes and TAQ polymerase according to manufacturer's recommendations.

The expected TH band was present in the sample of immature hNT neurons harvested immediately after 6 weeks of RetA, as well as in the sample of mature purified hNT neurons that were aged in culture for 5 weeks after mitotic inhibitor treatment. The TH band was not detected for either the uninduced NT2 precursor cells or the 24hr RetA-induced cultures. These results are consistent with other observations that TH is not expressed in the precursor cells or the NT2 cells early in the induction period (personal communication, Virginia Lee). TH begins to appear only after several weeks of RetA induction as the neurons develop and continues to be produced during the differentiation of hNT neurons. Similar results were obtained for the PCR analysis of the dopamine D1 and D2 receptor expression.

### Example 2

### Western Blot Assay for TH Expression

Protein samples and cell extracts were denatured in SDS sample buffer containing β-mercaptoethanol and electrophoresed on a Laemmli SDS polyacrylamide gel which separates the proteins by size. The proteins were then transferred to a nitrocellulose membrane by electro-blotting and the specific protein of interest was immunodetected by reacting to a primary antibody which is developed using the appropriate secondary antibody enzyme conjugate system.

Samples were prepared by harvesting cells with trypsin/EDTA solution, or by scraping, and resuspending in media. An aliquot of cells was taken for viable cell count, and total viable cells were determined. The remaining cells were centrifuged and washed, and the resulting cell pellet were quick-frozen and stored at −180° C. Samples were thawed and lysed in cold RIPA buffer at a concentration of 10⁶ cells in 10 μl of buffer, an equal volume of 2X SDS reducing sample buffer added, and heated in boiling water bath for 5 min and then placed on ice.

Samples of 20 μl, equivalent to 10⁶ cells per lane, were loaded on SDS polyacrylamide gels. The TH protein standard (STI, Catalog No. P-20233) at 500 pg/ sample and the prestained protein molecular weight marker samples were prepared in SDS reducing sample buffer. The gel was electrophoresed until protein markers were well separated and bromophenyl blue dye had run off the bottom of the gel. The protein samples were transferred from the gel and immobilized on a nitrocellulose membrane by electroblotting in a Western transfer chamber. The Western blot was blocked overnight in PBS containing 2% dried milk and incubated with anti-TH monoclonal (Boehringer-Mannheim clone 2/40/15) in PBS-Tween containing 1.0% BSA. The blot was incubated at room temperature with biotinylated goat anti-mouse secondary antibody (1:1000 BM) invitro and then with Streptavidin conjugate (1:2000). The blot was washed in PBS-Tween and developed using the insoluble alkaline phosphatase substrate (Sigma). The TH specific bands are visualized within 1-2 min. and appeared in the 55 to 60 Kd size range. The sensitivity of the assay was determined to be approximately 50 pg/lane using serial dilutions of the TH-protein standard (STI) ranging from 5 ng to 20 pg.

TH levels were confirmed and quantified by Western blot assay. In order to confirm and quantitate the levels of TH expressed in different samples, a Western blot assay was developed. Several monoclonal antibodies were evaluated for detection of TH on blots containing human cell extracts and rat TH standard (STI). The anti-TH monoclonal 2/40/15 (Boehringer Mannheim) was found to detect reproducibly 50 to 100 pg of TH per lane. Initial estimates of the amount of TH protein expressed were determined using the Western blot assay and purified rat TH (STI) as standard. Analyzed samples of hNT neurons contained a range of cells from 2×10⁶ to 2×10⁵ per lane. The intensity of the TH-specific band was compared to samples containing 500 pg to 50 pg of the TH standard. The results of this analysis indicated that approximately 200-500 pg of TH protein are present in extracts containing 10⁶ hNT neurons. A similar analysis was performed using human mesencephalic samples ranging from 10⁶ to 10⁵ per lane. Equivalent levels (200-500 pg/10⁶ cells) were detected in the hNT neurons compared to the human mesencephalic tissue.

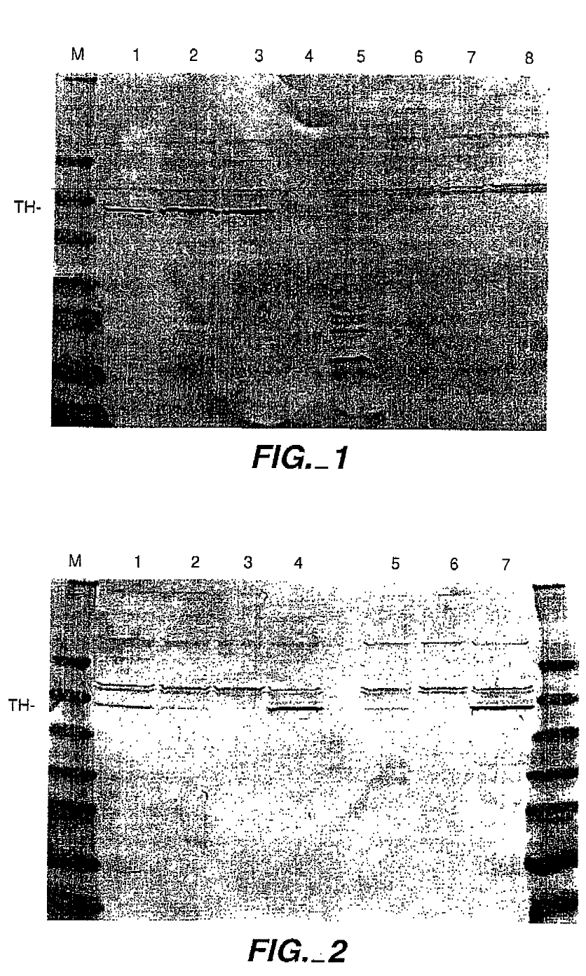

The commercial process of producing hNT neurons involves inducing the NT2/D1 precursor cells with RetA for 6 weeks. The resulting culture contains 10% neurons and 90% non-neuronal accessory cells, which are next mitotically inhibited. The hNT neurons are selectively harvested, leaving the accessory cells behind and the purified neurons are cryopreserved at 180° C. The levels of TH expressed in hNT neurons, NT-accessory cells, and NT2 precursor cells were compared by Western blot (FIG. 1). Only the sample of hNT neurons (lane 3) gave the expected TH band that migrated at 55-60 kd, and no TH-specific bands were detected in the accessory cell sample (lane 4) or NT2 precursor cell sample (lane 5).

### Example 3

### Preparations for Immunohistochemical Staining

Cells were fixed with 4% paraformaldehyde in 0.1 M NaPO₄, pH 7.4 and washed with phosphate buffered saline (PBS). The cells were incubated first in PBS containing 10% serum and 0.1% Triton X-100 for 30 min at room temperature and then overnight in the same solution containing the primary antibody. The cells were washed in PBS containing 1-2% serum and 0.1% Triton X-100, and then incubated with the biotinylated secondary antibody. Cells were washed as described and placed in 1:500 Streptavidin HRP for 2 hr. Preparations were developed with DAB (following manufacturer's instructions). Photomicrographs were taken, and immunoreactivity of dopaminergic neurons assessed as described below.

### Example 4

### Enhancing Dopaminergic Properties of hNT Neurons

A series of studies evaluated the dopaminergic potential of other neuronal precursor cell lines, the optimal time for RetA induction of TH during the differentiation of the NT-Neurons, and the process for stabilization of TH during replate purification.

4.A. Optimal clone selection

Other neuronal precursor cell lines were evaluated for their potential to produce neurons with dopaminergic properties. The 10 sister clones to the NT2/D1 (p51) production cell line were obtained from the Wistar Institute and analyzed for growth and production of neurons.

The four best growing clones were evaluated for production and yield of neurons which were further tested for the presence of TH by Western analysis. Only the neurons from the NT2/B9 and D1(P30) clones expressed TH at reproducibly detectable levels on Western blots and also were determined using the HPLC assay to produce HVA at levels similar to the hNT Neurons.

4.B. The Effect of maturation in replate of hNT-Neurons on TH expression

A study examined the effects of in vitro maturation and extended replate of hNT neurons on TH levels. After the hNT Neurons have developed during the 5 to 6-wk RetA induction, cultures are routinely replated and treated with mitotic inhibitors in the process of purifying the neurons (see Example 1). The hNT Neurons were maintained in culture and allowed to mature either as a replate-I or as an enriched replate-II culture. To optimize TH expression, neurons were purified after culturing under different replate conditions, and the levels of TH compared in the Western Blot assay. Samples were prepared from purified hNT Neurons that had been treated with inhibitors for 7 days as replate-I or replate-II cultures, and then maintained in growth media for a total of 1, 2, or 3 weeks of maturation. Extracts of the purified neurons were analyzed by Western Blot (FIG. 2). The level of TH expression decreased dramatically with maturation in culture, and it was no longer detectable after 2 weeks in replate-II or after 3 weeks in replate-I. The levels of TH found in the replate-I neurons not only were significantly higher, but also were expressed for a longer period. Control Western Blots (not shown) were developed for each assay using an anti-Tau monoclonal to confirm that 1) equivalent numbers of hNT neurons were loaded, and 2) the samples were not degraded.

4.C. Optimization of RetA induction for TH expression in Neurons

TH expression paralleled the early development of neurons and was evident by 3 to 4 weeks of RetA induction. Since the preliminary results showed that the TH expression level had been greatly reduced upon maturation of hNT neurons for 2 to 3 weeks after replate, a strategy was designed to determine if more TH was produced by less matured neurons. Possibly the hNT neurons that were produced after 5-6 weeks of RetA induction, which was optimal for the yield of cholinergic neurons, have been committed to down-regulate TH. To optimize production of neurons for expression of TH and dopaminergic properties, a time course of RetA induction was performed; and the TH levels in purified replate-I neurons from different RetA inductions were analyzed.

The NT2 precursor cells were induced with RetA for 4 weeks (DA neurons) or 5 or 6 weeks (hNT neurons). The cultures were replated and after 24 hours mitotic inhibitors were added and maintained for 7 days. The cells were refed with growth media, and the neurons harvested either after 1 day or after an additional 7 days. The extracts were prepared for denaturing SDS-PAGE, and samples containing the equivalent of 10⁶ cells/lane were transferred to Western blots.

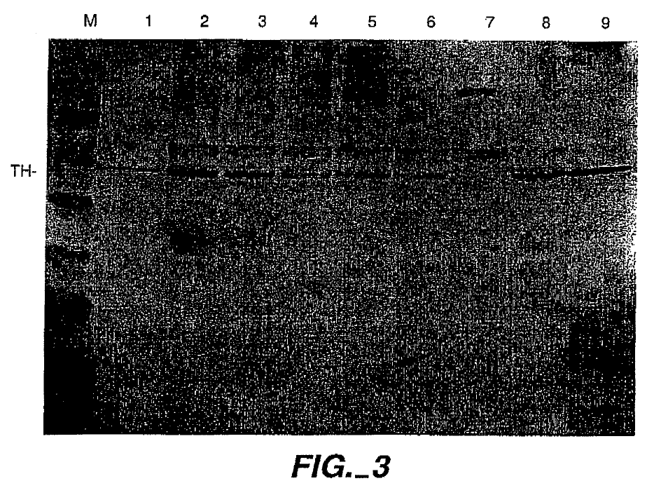

The dramatic effect of RetA induction times on TH expression levels in the neurons is shown in FIG. 3. The expression of TH was found to be the highest in the DA neurons that were purified from the 4-week RetA Induction (Lane 2). The TH levels decreased significantly in purified hNT neurons RetA induced for 5 and 6 weeks (compare Lane 2 to Lanes 3 and 4). The loss of TH expression becomes even more evident in the 2-week matured DA neuron (4-wk RetA) samples (Lane 5) compared to hNT neurons (5- and 6-week RetA) (Lanes 6 and 7). These results demonstrate that there is an optimal RetA induction period of greater than 3 but less than 5 weeks, perhaps peaking at 4 weeks. These results also confirm that subsequent maturation in vitro reduces Th expression, even in the high-expressing immature DA neurons (4-week RetA).

### Example 5

### Comparison of DA and hNT Cryopreserved Neurons after Thawing and Plating in Culture for 5-Days

To further characterize dopaminergic neurons, cells were treated with retinoic acid for 4 weeks (DA neurons) or 5 weeks (ANT neurons), were cryopreserved, stored frozen, thawed, and then cultured for 5 days. Additional testing was performed to determine if the DA neurons had in common additional biochemical attributes of the substantia nigra (SN) dopaminergic (DA) neurons, including tyrosine hydroxylase (as discussed above), dopamine membrane transporter for reuptake of dopamine from the synaptic cleft, D2 dopamine receptor for regulating dopamine release and aldehyde dehydrogenase (AHD2). AHD2 has been found in a subpopulation of dopaminergic neurons of the mesostriatal and mesolimbic system shortly after the appearance of TH but not in other dopaminergic neurons.

DA neurons and hNT neurons (Layton Bioscience, Inc., Atherton, Calif. ) had been stored at −180° C. prior to use. The cells were thawed as described above, resuspended in the medium containing DMEM (Gibco) and 10% fetal bovine serum (Gibco), and were plated on poly-L-lysine coated 8-well chamber slides at a concentration of 100,000 cells/cm². After 24 hr the plating media was switched to DMEM: F12 containing 0.1% ITS (Sigma) and gentamicin (50 μg/ml, Sigma). Cultures were maintained for an additional 4 days, then rinsed with 0.1 M PBS and fixed with 4% paraformaldehyde. However, some DA neurons were fixed after the initial 24 hr culturing to assess the effect of thawing on TH expression.

For immunochemistry, cultures were thoroughly washed in 0.1M PBS and incubated in 10% serum from the host secondary antibody with 0.03% Triton X-100 in PBS for 1 hr. Cultures were then incubated for 24 hr in the same solution containing one of the following: 1) primary antibody against TH (1:4000, mouse monoclonal antibody, INCSTAR, Stillwater, MN or 1:500, rabbit polyclonal antibody, Pel-Freez™, Rogers, AR), b) DAT (1:5,000, rat monoclonal or 1:500, rabbit polyclonal antibody, Chemicon, Temecula, Calif.), c) AHD-2 (1:5000, rabbit polyclonal antibody, courtesy of Dr. Ron Lindahl, University of SD) d) D2 receptor polyclonal antibody (1: 1000, rabbit polyclonal antibody, Chemicon, Temecula, Calif.) or a mixture of primary antibodies, including antibodies to TH combined with an antibody to DAT, D2 or AHD-2.

For single staining, the slides were washed with PBS and incubated for 1 hr in the appropriate biotinylated secondary antibody (1:200, Vector, Burlingame, Calif. ). The antibody complex was developed using avidin-biotin kit (ABC-Elite kit, Vector), and the final product was visualized either by using 3,3′-diaminobenzidine (DAB; ImmunoPure Metal Enhanced DAB, substrate kit; Pierce, Rockford, Ill. ) or VIP (Vector, peroxidase substrate kit, Burlingame, Calif. ). For immunofluorescent staining, TH was visualized using fluorescein isothiocyanate (FITC) conjugated to goat anti-mouse IgG (1:500, Alexa™, Molecular Probes, Inc., Eugene, Oreg.) or rhodamine conjugated to goat anti-rabbit IgG (1:200, Jackson ImmunoResearch), AHD-2 and DAT were visualized using rhodamine conjugated to goat anti-rabbit IgG (1:200). Finally slides were rinsed in 0.1 M PBS and cover-slipped using 95% glycerol or Vectashield (Vector). For control sections, one or both of the primary antibodies were omitted. The enzyme-linked immunostained slides were examined using an Olympus BH-2, while immunofluorescence-stained slides were analyzed and photographed using the Olympus BX 40 and BX 60.

Prior to cell counts, 8-well slides were stained with the appropriate primary antibody and visualized with DAB. Through 20X objective and a photographic frame (field =0.3 mm²), 16 predetermined sites per well (4-8 wells/plating/marker) were viewed to count the number of labeled and unlabeled cells. Percentages were determined as a ratio of labeled cells/total cells multiplied by 100. The mean values ± SEM for every marker were determined from three independent cultures. The differences between the dopaminergic markers between DA and hNT neurons were compared using Student's t-test.

The co-localization of TH with other dopaminergic markers was evaluated from fluorescently labeled slides viewed through single or double fluorescent filters. The co-expression of two markers was determined in randomly selected fields (n=30-40/plating), under 40X magnification. Cells that were positive for DAT, D2, or AHD-2 alone or positive for both markers (TH/DAT, TH/D2, or TH/AHD-2) were recorded.

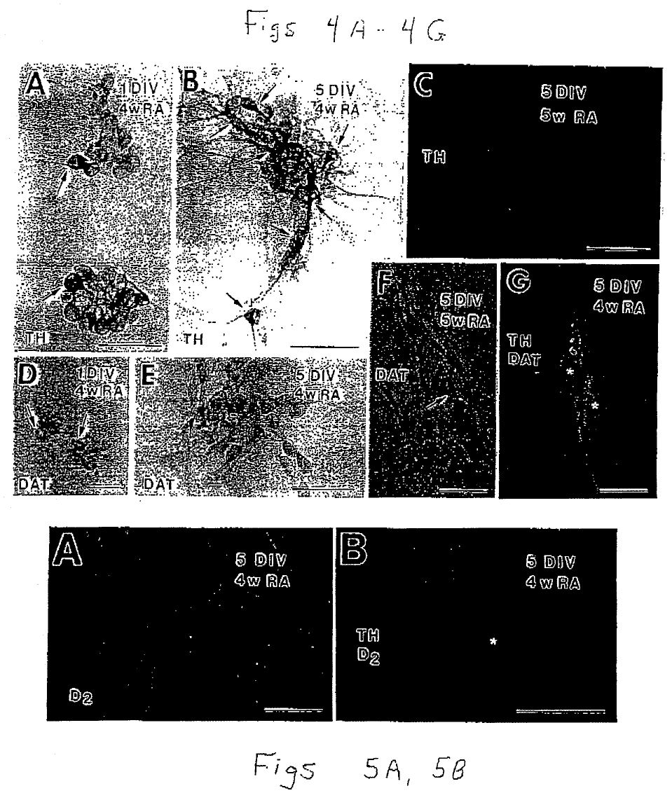

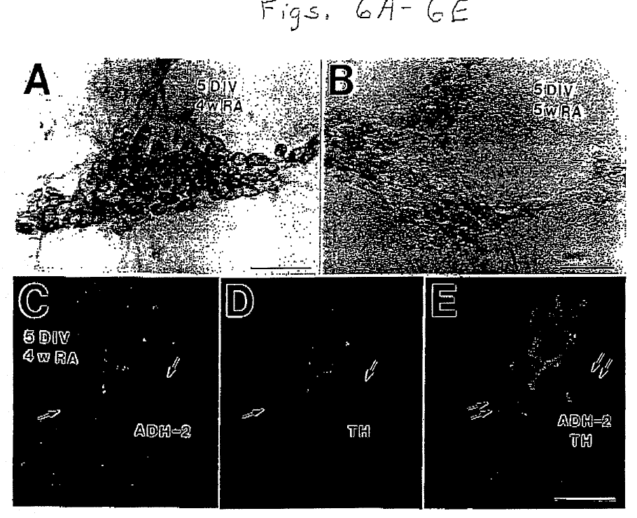

24-hr plated cells had many clumps of small round cells evenly spread over the culture dish. Numerous cell bodies stained for TH; but there were only a few extended, short processes that were not very prominent (FIG. 4A). Representative cultures contained 44.3-64.9% TH+ neurons. DAT was also found and had a more variable staining because of diffuse label in some cells and a more common punctate/granular appearance in cell somas and processes (FIG. 4D).

After 5 days in vitro (DIV), cell morphology substantially changed. Individual cells and clusters of varied sizes revealed TH+ positive cell bodies and long, branching processes spread toward neighboring clumps (FIG. 4B). Quantitatively among 5 DIV cultures, the percentage of TH+ cells varies between 33.3% and 87.2% which was not significantly different from TH+ cells in 1 DIV cells. However, TH expression did differ significantly (p<0.01) between DA neuron and hNT neuron cultures (58.7% and 14.8%, respectively) (FIGS. 4B and 4C). Interestingly, the percentages of DAT+, D2+, and AHD-2+ were equally high for DA and hNT neurons (79.9% to 91.2%). FIGS. 4E and 4F show DAT staining of DA and hNT cells, respectively. See Table 1.

TABLE 1
Dopaminergic Phenotype of DA and hNT neurons (5DIV)

| RA treatment | TH | DAT | D2 | AHD-2 |
|---|---|---|---|---|
| DA neurons | 58.7 ± 3.7 | 79.9 ± 2.2 | 91.2 ± 3.9 | 82.7 ± 9.2 |
| | (5419) | (3042) | (1192) | (2102) |
| hNT neurons | 14.8 ± 2.3 | 79.2 ± 2.9 | 89.9 ± 0.8 | 81.9 ± 1.9 |
| | (2176)* | (2631) | (1229) | (3140) |

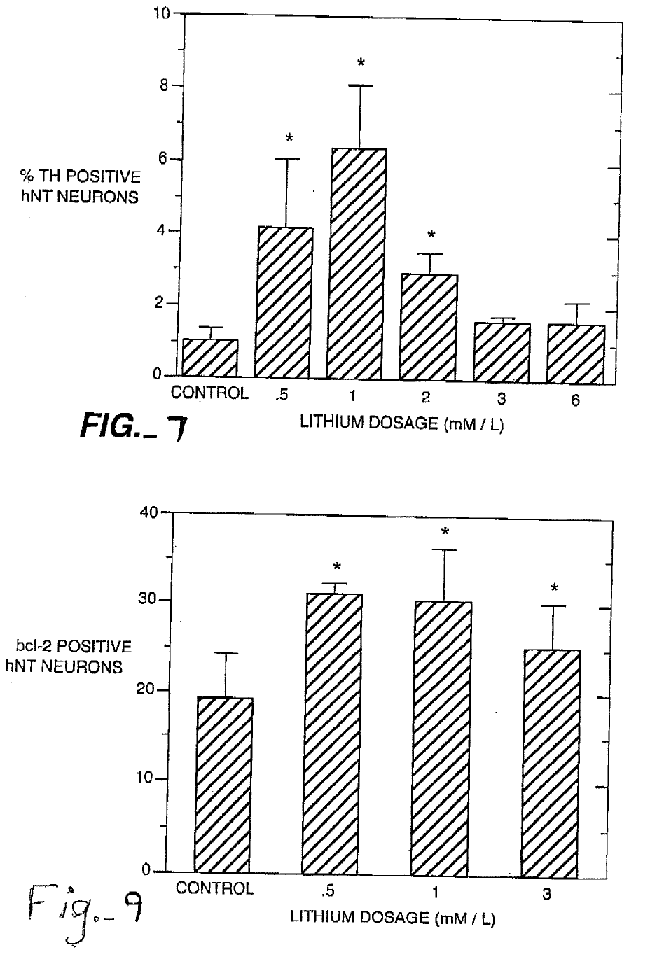

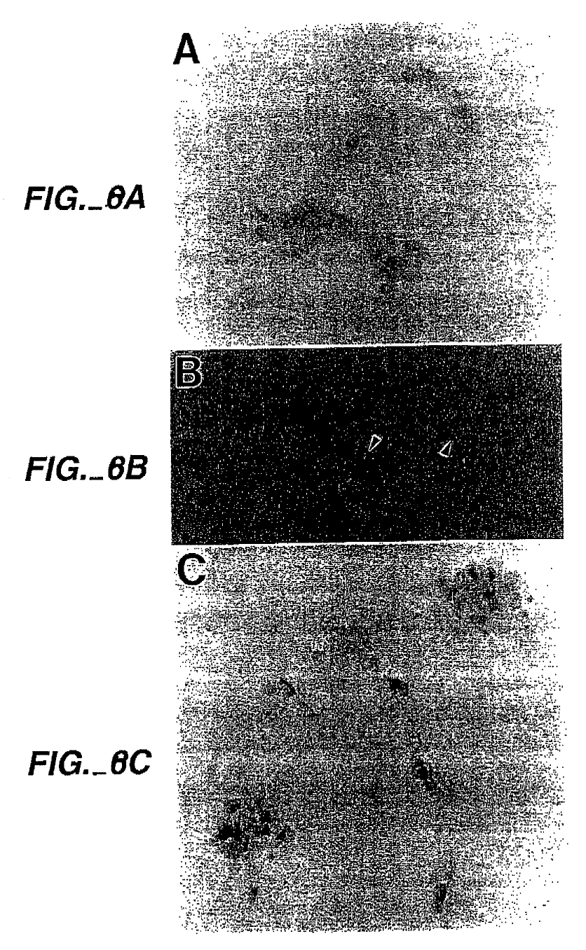

Nearly all TH+ neurons (93%) also were DAT+. Moreover, 53% of all D2+neurons also stained for TH, indicating TH+ cells also have D2 dopamine receptors to regulate dopamine release. D2 staining alone of DA neurons is shown in FIG. 5A; combined TH and D2 staining of DA neurons is shown in FIG. 5B. Virtually all TH+cells were also AHD-2 positive, indicating that DA neurons had a phenotype typical of the cells involved in Parkinson's disease (a subpopulation of dopaminergic neurons of the mesostriatal and mesolimbic system). FIGS. 6A-6E show AHD-2 staining of DA and hNT cells with and without TH staining. FIG. 6A shows 5 DIV DA neurons, more of which stained for ADH-2 (chromogen DAB) than did the hNT neurons in FIG. 6B (chromogen VIP). For FIGS. 6A and 6B the bar is 50 μm. FIG. 6C shows ADH-2+ DA neurons (red fluorescence, arrows) visualized by a rhodamine-conjugated secondary antibody. FIG. 6D shows TH+ DA neurons visualized by a secondary antibody conjugated to fluorescein. FIG. 6E shows double-labeled (TH+/AHD-2+) DA neurons. For FIGS. 6C-6E the bar is 25 μm.

In conclusion, these cells have all the necessary cellular machinery to produce functional dopaminergic neurons and therefore are a better choice as an alternative tissue source to fetal ventral mesencephalon than many other dopamine-producing cells. TH alone is not a sufficient marker for a dopaminergic cells since TH also participates in synthesizing other catecholamine neurotransmitters (epinephrine and norepinephrine). Dopamine neurotransmission also requires presynaptic release of dopamine and its reuptake through the sodium-dependent DAT. Thus, the DAT activity determines the synaptic concentration of dopamine and the level of dopamine receptor stimulation.

5A. TH Expression of hNT Neurons treated with Lithium Chloride

A small-scale dose range study analyzed whether therapeutic (0.5-1.0 mM) and clinically toxic (2.0-6.0 mM) concentrations of LiCl enhanced TH expression in cells treated for 6 weeks with retinoic acid and then LiCl. The control and lithium-treated cultures (0.5, 1.0 and 3.0 mM) were scored for TH immunoreactivity, which was expressed as the number of TH-positive (TH+) cells/well. For TH immunohistochemistry the cells were rinsed in 0.1 M phosphate buffered saline (PBS), fixed in 4% paraformaldehyde, and then again rinsed in PBS. Before the primary antibody was applied, the cells were incubated in blocking serum (10% normal horse serum, in 0.02% Triton X-100 in 0.1 M PBS, pH 7.4) for one hour and then incubated overnight in monoclonal antibody against TH (1:4000, INCSTAR, Stillwater, Minn.). The next day, secondary antibody—biotinylated horse antimouse (1:300, Vector, Burlingame, Calif.)—was applied for one hour. The antibody complex was developed using avidin-biotin complex (ABC-Elite kit; Vector), and the developed product was visualized by using DAB (Pierce, Rockford, Ill.). To determine the percentage of cells labeled by a particular antibody in representative experiments, the numbers of labeled and unlabeled cells were assessed in a blind-coded manner using a 20X objective and a photographic frame. The frame was placed in 16 predetermined sites per well. This test was performed in duplicate.

Results are summarized in FIG. 7. Of controls, TH+ neurons represented only 1% of the entire population of hNT cells. After treatment with 0.5 and 2.0 mM LiCl, almost 5% of hNT cells became TH+. The TH+ cells constituted nearly 7% of hNT cells treated with 1.0 mM of lithium, approximately a five-fold increase. At higher concentrations (3.0 and 6.0 mM lithium), TH+ cells represented fewer than 2%. Incubation of hNT cells with LiCl increased the size of the TH neurons as well as the length and number of processes. Some cells revealed extremely long TH+ processes. Intensely stained TH+ cells had well developed processes that are typical of mature neurons. In summary, the results show that 0.5, 1.0, and 2.0 mM concentrations significantly enhanced the expression of TH in hNT neurons. (Zigova T, et al., LiCl induces the Expression of TH in hNT Neurons, Exp Neurol 157(2): 251-8, 1999).

Fresh and thawed DA neurons were compared. “Fresh Cells” had undergone Replate treatment (see above) and were not frozen. Prior to freezing, other cells underwent Replate I treatment. Control and lithium-treated cells (fresh or frozen) were cultured for five days and scored for TH immunoreactivity (see above method). The TH-immunostained slides were counterstained with propidium iodide to identify dead cells. Total numbers of TH+ and propidium iodide-positive (PI+) neurons were counted in control and lithium-treated cultures from standardized fields at 20X magnification, as described above.

TABLE 2
TH Expression in cultured (fresh Replate II) DA neurons

| LiCl Dose | TH+ cells | Unlabeled Cells | Total Cells |
|---|---|---|---|
| Control | 256 (56.1%) | 209 | 465 |
| 1.0 mM | 318 (85.3%) | 53 | 371 |
| 3.0 mM | 452 (76.8%) | 138 | 590 |

TABLE 3
TH Expression in cultured (thawed) DA neurons

| LiCl Dose | TH+ cells | Unlabeled Cells | Total Cells |
|---|---|---|---|
| Control | 649 (70.4%) | 272 | 921 |
| 1.0 mM | 846 (79.1%) | 224 | 1070 |
| 3.0 mM | 623 (70.4%) | 262 | 885 |

These results indicate that 4-week RetA induced cultures of DA neurons have significantly higher number TH+ cells (56-70%) than the hNT neurons induced for six weeks (1%). These results are consistent with the Western analysis that also shows higher TH levels in DA neurons compared to hNT neurons. In addition, Western analysis indicates that the levels of TH in fresh Replate II cultures were lower than those found in frozen neurons (Table 3). Exposure to 1.0 mM LiCl significantly increased the number of TH+ cells to 80-85%. When no primary antibody was added to the negative control cultures (primary delete), the control cultures were immunonegative.

Photomicrographs (FIGS. 8A-8B) show representative 5 DIV DA neurons that had been induced for four weeks with RetA. FIG. 8A is a control culture of DA neurons immunostained with antibodies to TH which had a significantly higher number of TH+ cells in comparison to controls treated for six weeks with RetA (See FIG. 7). FIG. 8C is a control culture of DA neurons immunostained with antibodies to bcl-2 to demonstrate the co-localization of an anti-apoptotic gene with TH expression.

### Example 6

### Viability of hNT Neurons Treated with Lithium Chloride

The effect of LiCl on hNT viability was assessed using the Trypan blue exclusion technique (blue dead cells) and a double-staining procedure using fluorescein diacetate (FDA) and propidium iodide (PI), according to Jones and Senft (J Histochem Cytochem 33:77-80, 1985). The hNT cells were seeded in 10 μg/mL poly-L-lysine-coated 8-well chamber slides at a concentration of 100,000 hNT neurons/cm² in a DMEM medium supplemented with 10% FBS and 50 μg/mL gentamicin (Sigma). Plated cells were maintained in a humidified CO₂ incubator (5% CO₂, 90% air) at 37° C. After 24 hours, the media was changed to DMEM:F12 containing 0.1% ITS (Sigma), gentamicin, and LiCl (Sigma) at 0, 1.0 and 3.0 mM concentrations. The cells were incubated for an additional four days. Then, trypan blue was added to the medium. In three random fields from control and both lithium-treated cultures, the numbers of viable and dead cells were counted.

Viability of hNT cells in 1.0 mM and 3.0 mM LiCl was 71.55% and 70.15%, respectively, compared to untreated hNT control cell viability of 74.22%. Thus, 5 days of LiCl treatment did not significantly change the survival of hNT cells.

### Example 7

### Lithium Induction of bcl-2 Expression

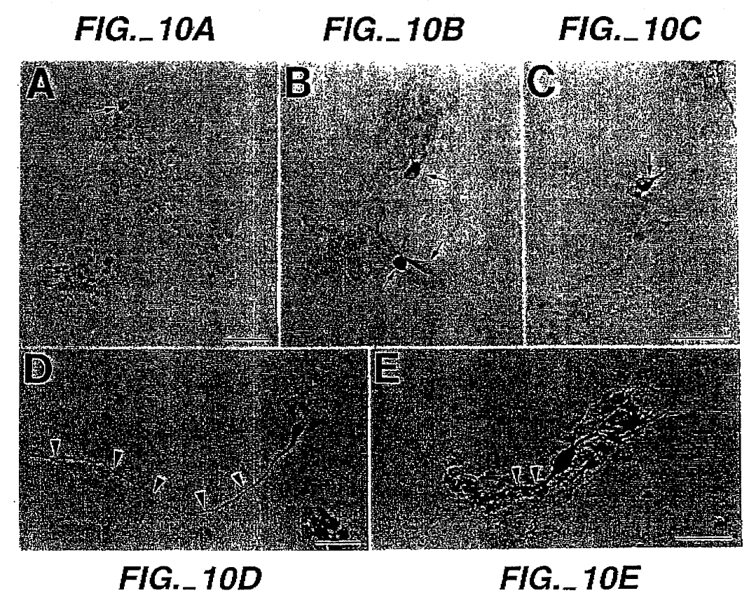

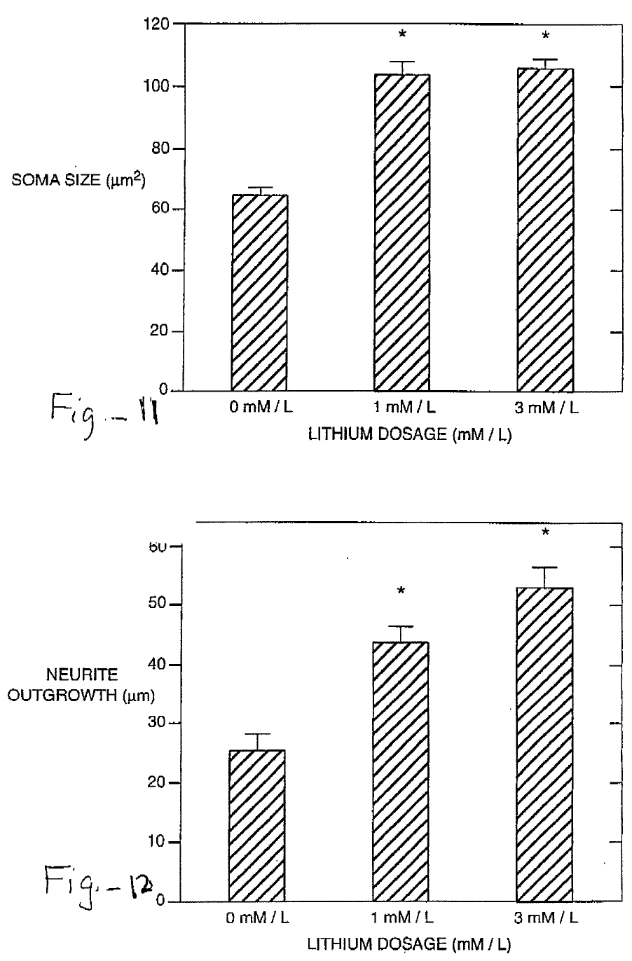

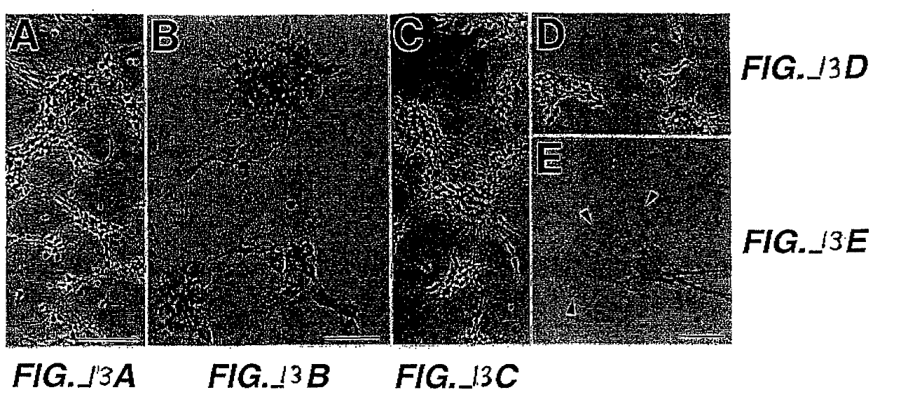

Because the proto-oncogene bcl-2 has been shown to protect a variety of cell types from programmed cell death, it is often considered an inhibitor of apoptosis (Sentman et al. Cell 67(5): 879-88, 1991). Lithium-treated cells were tested for the involvement of bcl-2 that could help protect hNT neurons from apoptosis. First, the immunocytochemical expression of bcl-2 protein in hNT cells cultured for 5 days with 0.5 and 3.0 mM LiCl was evaluated. Immunostaining was performed as described for TH (above), except that the monoclonal antibody to bcl-2 (Ab-1, 1:50, Calbiochem, Oncogene Research Products, Cambridge Mass.) was used. FIG. 10 summarizes the effects of different doses of LiCl on bcl-2 immunostaining. The number of bcl-2+cells per well versus total number of cells in experimental (0.5 and 3.0 mM) and control groups were compared. Among control cells, 19.14% (288/1504) were bcl-2+; among 0.5 mM lithium-treated cells, 31.62% (278/879) were bcl-2+; and among 3.0 mM lithium-treated cells, 29.50% (562/1903) were bcl-2+. These results indicate that lithium enhances bcl-2 expression in hNT cells and thus may act as a neuroprotective agent.

### Example 8

### Effects of Lithium Chloride on hNT Cells

In the present study, lithium chloride in several doses was tested for its ability to induce the expression of tyrosine hydroxylase (TH) in hNT neurons after 5 and 10 DIV. The data suggests that hNT cells respond to lithium exposure and may serve as a continual source of TH-expressing neurons in new therapeutic approaches to degenerative brain disease.

hNT neurons (Layton Bioscience, Inc., Atherton, Calif.) were cultured with LiCl as described in Example 5A. Immunochemistry was performed as described in Example 3.

8A. Effect of Lithium on TH expression in hNT Neurons

To investigate the effect of various doses of LiCl on tyrosine hydroxylase expression in 5 DIV hNT neurons, we determined the number of unlabeled and TH+ neurons in a blind coded manner using a 20X objective and a photographic frame (field=0.3 mm²). The frame was placed in 16 predetermined sites per well. The counts of TH+neurons in lithium-treated cultures were compared with counts obtained from cultures treated with NaCl or KCl. The dose responsive tests were analyzed using a one-way analysis of variance (ANOVA) followed by post hoc comparisons using Bonferroni-Dunn analysis.

Photomicrographs of representative control and lithium-treated 5 DIV hNT neurons and TH immunostained are shown in FIG. 10A, in which control culture of hNT neurons reveals few TH+ cells (arrow). The Bar shows a scale of 50 μm. (FIG. 10B and 10C) show hNT cells cultured with 1.0 mM (FIG. 10B) and 3.0 mM (FIG. 10C) lithium chloride; arrows point to individual TH+ neurons. In lithium-exposed cultures more TH+ cells with longer TH-immunoreactive processes (arrowheads) were present. FIGS. 10D and 10E show the representative morphology of TH+ hNT cells treated with 1.0 mM (FIG. 10D) and 3.0 mM (FIG. 10E) of lithium chloride. Even though only cultures supplemented with 1.0 mM dose of lithium chloride increased the overall yield of TH+ hNT neurons, both the 1.0 mM and 3.0 mM doses promoted morphological development. Frequently, intensely TH+ cells with well-developed processes (arrowhead), typical of mature neurons were found.

In order to determine whether the induction of TH expression would be sustained if lithium chloride administration were terminated, the number of TH+ neurons on day 10 was assessed. The control cultures and cultures exposed to 1.0 mM lithium chloride for 5 days, received new media lacking LiCl and were maintained until day 10. Control cultures revealed a relatively low percentage of TH+ cells (less than 2%), while cultures exposed to 1.0 mM lithium chloride displayed the values comparable to values obtained on day 5 (ranging between 5-7%). This indicated that hNT cells maintained the expression of TH even though the inducing agent LiCl was absent from the media after day 5.

In a parallel series of experiments, the effect of NaCl or KCl (1.0 mM concentrations) was compared to that of LiCl (1.0 mM) on TH expression in 5-day cultures of hNT neurons. The numbers of TH+ hNT cells in cultures treated with either NaCl or KC1 were not significantly different from values obtained in control (not supplemented with lithium). From these results it could be concluded that lithium and not the chloride (salt) was responsible for the induction of TH expression in hNT neurons. The findings of this study demonstrated that LiCl induced the expression of TH in cultured hNT neurons. First, it was shown that the induction of the TH expression in hNT cells was dose-dependent. Second, the effect of an optimal dose of LiCl treatment in the optimal dose (1.0 mM) was sustained even after the treatment with LiCl was discontinued.

Previous reports have demonstrated the induction of TH expression in cultures of primary brain neurons (Du X, Stull N D, lacovitti A, Brain Res 680: 229-233, 1995); however, the importance of finding alternative dopaminergic sources prompted the idea to induce the expression of TH in other non-TH expressing neurons. Co-culturing hNT cells with Sertoli cells also clearly induced TH production (Othberg A I, Willing A E, Cameron D F, Anton A, Saporta S, Freeman T B, Sanberg P R, Cell Transplant 7: 157-164, 1998). Together with the above mentioned findings, the present results showing the induction of TH expression after lithium treatment support the idea that this cell line, previously low in intrinsic TH expression, can be converted into significantly high levels of TH-expressing cells.

In the present study, lithium, an effective psychotherapeutic agent induced the expression of TH in hNT cells. Not wishing to be bound by a particular theory, the increased TH expression can be due to increased TH synthesis and/or increased TH activity, both of which are mediated by signal transduction pathways including protein kinase C (Zigmond R E, Schwarzschild Mass., Rittenhouse Ark., Ann Rev Neurosci 12:415-461, 1989).

8B. Effect of Lithium on Size and Neurite Outgrowth of hNT Neurons

The same series of slides immunostained for TH were employed to measure the soma size (μm²) and neurite outgrowth (μm) of appropriately 50 TH+ cells per representative culture experiment using a computerized image analysis program (Image-Pro Plus, Media Cybernetics, Inc., Silver Springs, Md.) at 20X objective. The results in the morphological assessment study are reported as mean ±SEM and were analyzed using Student's t-test. The size of the TH+ cell bodies increased significantly (p<0.01) after application of 1.0 and 3.0 mM dose of lithium chloride, ranging from 33.8 μm²to 103.3 μm² (mean=64.1±2.5 μm²) in the control group and from 53.09 to 183.3 μm² (mean=103.2±2.7 μm² ) and from 61.4 to 165.8 μm² (mean=104.8±3.2 μm²) in 1.0 and 3.0 mM lithium-treated groups, respectively (FIG. 11). Soma sizes in 5 days in culture NaCl or KCl-treated cultures were not significantly different from control (FIG. 11).

The second parameter characterizing the effect of LiCl on the development of TH+ hNT cells was neurite outgrowth. The length of processes in controls has a mean of 25.02±2.9 μm, while in both groups exposed to LiCl significantly (p <0.01) longer processes were found (FIG. 7E, 7D). In cultures treated with 1.0 mM dose of LiCl, the lengths ranged between 12.2 μm and 87.3 μm (mean=43.4±2.8 μm) and in the group treated with 3.0 mM concentration the lengths varied between 20.3 μm and 128.1 μm (mean=52.9±3.8 μm). Neurite outgrowth in NaCl and KCl-treated 5 days in cultures did not significantly differ from control values (FIG. 12). These results clearly demonstrated that morphological development was significantly enhanced in TH+ hNT cells treated with both LiCl concentrations.

Soma size and neurite outgrowth were also measured in hNT cells maintained in culture for 10 days and treated for 5 days with the most effective dose of LiCl (1.0 mM). The mean soma size of TH+ hNT cells was significantly larger (102.8±2.5 μm²) (p<0.01) than in 5 days in cultures, but did not differ significantly from the mean value of the lithium-treated group (103.2±2.7 μm²). The mean length of neurite processes in controls was 24.8±2.4 μm which was not different from younger (5DIV) control cultures but significantly different from 10 day LiCl-treated group (55.5±5.1 μm). In addition, as a result of LiCl treatment, numerous TH+ cells revealed multiple branching processes with varicosities. Collectively, these results suggested that TH-converted hNT cells responded to LiCl treatment by enhancing morphological maturation at both time points studied.

Morphometric analysis revealed that TH+ cells in cultures exposed to lithium resulted in significantly enlarged soma size and longer neurites, as well as a higher degree of neuronal complexity. Taken together, our results suggested that the most effective concentration of lithium (1.0 mM) was adequate to induce TH expression and morphological development of cultured hNT neurons.

8C. Effect of LiCl on Viability of hNT Neurons

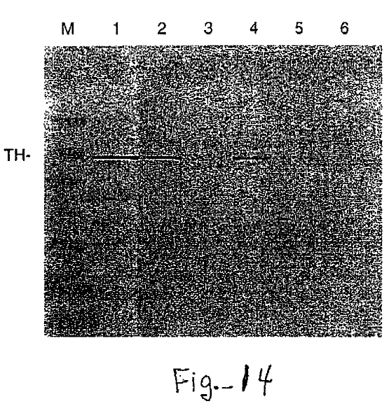

The effect of LiCl on survival of hNT neurons was also evaluated from cultures fixed and immunostained for neuronal marker, growth-associated protein (GAP-43). The actual counts were obtained by placing the photographic frame of the microscope over five randomly chosen fields (field size=0.2 mm²) in each well at 200 ×magnification. The mean number of GAP-43-positive cells per field was calculated from 4 wells per condition. The immunostaining was selected-as further confirmation of hNT's neuronal phenotype and to facilitate the neuronal counts. Morphologically, GAP-43+ hNT neurons usually exhibited round or oval perikarya and neurites including growth cones (FIG. 10). In FIG. 13B, higher magnification shows that virtually all cultured hNT cells are GAP43+, and thus have a neuronal phenotype. The scale is 50 μm.

Independent of survival time or lithium dose used, hNT neurons were aggregated into tightly or loosely packed clusters frequently interconnected with each other (FIG. 13C, 13D). Low-magnification phase contrast photomicrographs of hNT neurons treated with 1.0 mM (FIG. 13C) and 3.0 (FIG. 13D) concentration of LiCl. In both experimental groups the aggregation pattern and morphological appearance of cultured hNT neurons was similar to cultures unexposed to LiCl treatment. The scale is 100 μm in FIGS. 13A, FIG. 13C, and FIG. 13D. FIG. 13E shows the appearances of GAP-43+ hNT neurons treated with 3.0 mM LiCl demonstrating GAP-43+ cell bodies with prominent growth cones (arrowheads) similar to those observed in cultures not supplemented with LiCl. The scale is 25 μm.

In addition, GAP-43 immunostaining facilitated the neuronal counts on these cultures, whose aggregates hampered the cell counting if unstained. Typically, in the 5 DIV hNT control cultures, the number of viable neurons varied between 110-140 per field that was similar to cultures receiving 1.0 and 3.0 mM LiCl. In control 10 DIV cultures and cultures treated with 1.0 mM LiCl, the individual counts per field ranged from 110-150 and in the group treated with 3.0 mM LiCl these varied between 100-140/ field. These counts were not statistically significantly different from the control values. When the mean number of neurons/field was used to calculate the total number of neurons per well in control cultures, it was shown, that there were about 50,000-54,000 cells/well at both 5 and 10 days. As the initial cell plating in all groups was 89,000 cells/well it suggests that there was an approximately 30-40% loss of cells caused by their detachment from the surface of the dish. Taken together, these findings indicated that the presence of 1.0 or 3.0 mM of LiCl had no deleterious effect on the survival of hNT neurons in vitro.

The second important finding of this study was that the most effective TH-inducing dose of LiCl (1.0 mM) was not detrimental to cultured hNT neurons. This dose is within the range of therapeutic concentrations (0.5-1.0 mM) (Johnson, Aust N Z J Psychiatry 21(3):356-65, 1987), and in addition to being employed in treatment of mood disorders, LiCl is a neuroprotective agent against a variety of neurological deficits. A neuroprotective effect of chronic LiCl administration on focal cerebral ischemia was recently shown by Nonaka and Chuang (Neuroreport. 9(9):2081-4,1998). The authors assumed that chronic LiCl-induced neuroprotective benefit is probably due to its ability to attenuate excessive calcium influx mediated by NMDA receptors. They also reported that chronic LiCl treatment (at therapeutically relevant concentrations of this drug −1.3 mM robustly protected cultured CNS neurons against excitotoxicity mediated by NMDA receptors (Nonaka et al., 1998). An anti-apoptotic effect of LiCl on cultured cerebellar granule cells has also been reported after application of anticonvulsant (Nonaka et al., J Pharmacol Exp Ther 286(1):539-47, 1998). Further studies demonstrating either enhanced expression of neuroprotective genes or decreased expression of pro-apoptotic genes in LiCl-treated hNT cells are necessary to confirm the possible neuroprotective effect of LiCl.

In summary, the present results indicate that LiCl does indeed increase the TH activity of hNT cells.

### Example 9

### Co-Culturing with Sertoli Cells to Stabilize Th Levels in DA Neurons

TM4 Sertoli cells (ATCC CRL-1715) were tested as a method to stabilize TH levels in DA neurons. The optimal conditions were used for the preparation of TM4 cells. T-25 flasks were seeded with 3×10⁶ cells in DF-5 media. After 24 hrs the cells were fed with DF-5/0.1X inhibitor solution for 48 hrs. The cells were then maintained in DF-5 media for a few days until DA neurons were plated. DA neurons were obtained by inducing NT2/D1 cells for 4 wks with RetA and separated from the accessory cells by gentle agitation of the flask. DA neurons (2×10⁶) were plated onto the confluent TM4 cells in duplicate flasks. Co-cultures were maintain in DF-5 media for 1 wk and then harvested and processed for Western blot analysis. The controls were DA neurons plated on lysine/laminin coated flasks, bulk DA neurons, and harvesting of the co-culture layer. FIG. 14 shows the results. Lane 1 contains 500 pg of TH, Lane 2 shows the results with bulk DA neurons, Lane 3 shows the results with DA neurons plated on lysine/laminin, lane 4 shows the results for DA neurons plated on TM4 cells. Lane 5 shows the results for the harvested co-cultured layer. Co-culturing the DA neurons for 1 wk maintained TH expression.

### Example 10

### Production of LiCl-Induced DA-Neurons

In small-scale cultures, expression of TH in the 4-week DA-Neurons was optimal after the neurons were replated in serum-free media containing 1 mM LiCl for 5 to 7 days. To determine the conditions for production of LiCl-induced DA-Neurons, a series of LiCl treatments were designated to evaluate conditions for optimal TH expression.

The purpose of this experiment was to determine the effect of LiCl treatment during the Replate I mitotic inhibition on DA neurons, which occurs just before harvest of the DA Neurons. Replate cultures were treated with 1 mM LiCl in the presence of mitotic inhibitors (FudR & AraC) for all 7 days of replate or only during the last three of the 7 days (prior to harvest). The 3-day LiCl treatment was also evaluated without mitotic inhibitors and in serum-free media (+ITS). After treatment the neurons were selectively harvested and processed, and TH levels were analyzed using Western Blots. The 7-day-LiCl DA Neurons contain about 50% higher levels of TH than the 7-day inhibitor-only control. Surprisingly, the 3-day-LiCl neurons also expressed levels of TH comparable to the control, with the uninhibited sample expressing somewhat more TH. The neurons harvested from serum-free media expressed significantly less TH. Serum-free neurons may not have developed as well and may have been contaminated with accessory cells.

DA neurons treated with LiCl expressed comparable levels of TH to those of DA neurons maintained in DF-5/Inh alone. DA neurons with 7 days of LiCl and with 3 days of LiCl had similar levels of TH. The weaker signals for some cells may be due to higher contamination with accessory cells. During harvest of some flasks, the accessory cell layer came off more rapidly than it did from other flasks.

### Example 11

### Stabilization of TH Expression in DA Neurons by Co-culturing

DA neurons were prepared as described above. Prior to co-culturing, flasks were prepared with the following cell types: bone marrow stem cells (BMSC; courtesy of Dr. Juan Sanchez-Ramos, University of South Florida, Tampa Fla.), TM4 Sertoli cells, and SF-126 human glioblastoma cells obtained from University of San Francisco Neurosurgery Tissue Bank, San Francisco, Calif.). As a control, DA neurons also were cultured on lysine/laminin.

Monolayer feeder cultures were allowed to reach confluency before plating the neurons. Due to the slow proliferation of BMSC, no mitotic inhibitors were added to the media. One day prior to co-culture, TM4 Sertoli cells were seeded at 2×10⁶ per T-25 flask in DF-5/Inhibitor.

Next, 2×10⁶ DA neurons were plated on duplicate flasks of TM4, BMSC, SF-126, and lys/lam, each flask receiving DF-5 media. One flask was harvested at 1 wk and the second harvested at 2 wk. DA neurons also were plated on one flask of BMSC cells, which was maintained 2 wk. The mature DA neurons were differentially harvested from the feeder cultures. Extracts were prepared from about 0.5×10⁶ DA cells, which were reduced, denatured, and analyzed for TH using a Western blot technique.

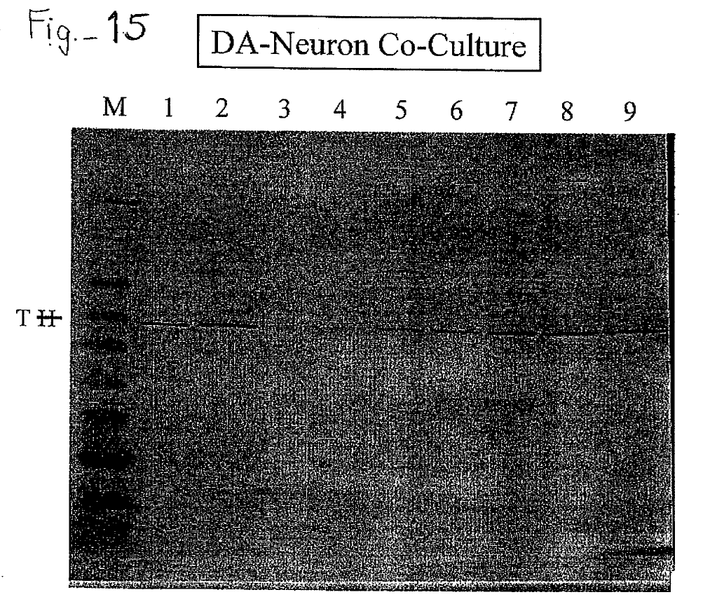

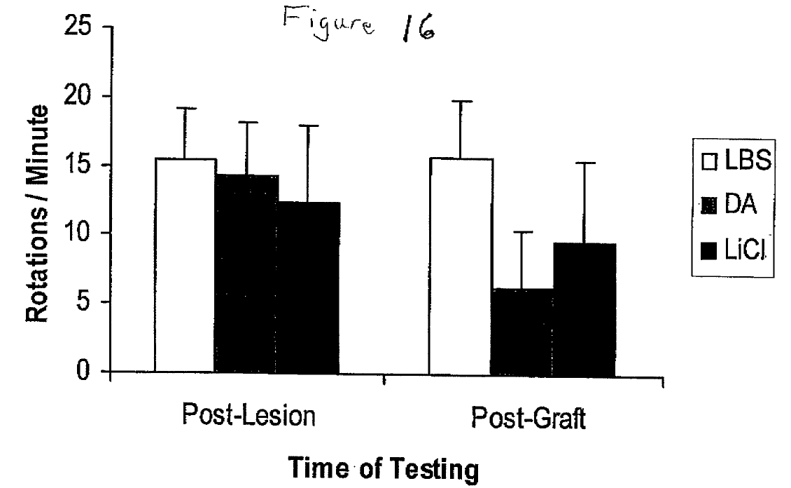

The levels of expressed TH are shown in FIG. 15. Lane 1 contains the control of 500 pg of TH, lane 2 shows TH expressed by fresh DA Neurons bulk harvested. Also shown are TH from DA cells cultured 1 and 2 wk with polylysine/Laminin (lanes 3 and 4, respectively), TM-4 cells cultured for 1 and 2 wk (lanes 5 and 6, respectively), BMSC cells for 2 wk (lane 7), and SF-126 cells for 1 and 2 wks (lanes 8 and 9, respectively).

Control lys/laminin cultures show how quickly the TH expression by DA neurons decreases in the absence of co-culturing. In contrast, TM4 and SF-126 definitely helped stabilized the TH levels, and to a lesser extent BMSC cells. These cells could be used in co-culture to produce an implantable DA neuron whose TH expression has been stabilized. Moreover, the DA neurons would be implanted with such cells that would then help maintain TH production in vivo.

### Example 12

### Comparison of DA and hNT Neurons in PD Rat Model

For 7 days before surgery, 27 female Wistar rats (Charles River, St. Constant, Quebec, Canada), weighing 200-225 g, were housed two per cage with food and water ad libitum and acclimatized for the animal care facility. All animal procedures were in accordance with the guidelines of the Canadian Council on Animal Care and the University Council on Laboratory Animals. Rats were anesthetized intramuscularly with 3.0 ml/kg of a ketamine-xylazine-acepromazine anesthetic mixture (25% ketamine hydrochloride; Ketalean, MTC Pharmaceuticals, Cambridge, Ontario), 6% xylazine (Rompun, Miles Canada, Etobicoke, Ontario); 2.5% acepromazine maleate (Wyeth-Ayerst Canada, Montreal, Quebec); in 0.9% saline and received two stereotactic injections of 6-OHDA (Sigma Chemical Company, Chicago, Ill.) (3.6 μg of 6-OHDA HBr/μl in 0.2 mg/ml of L-ascorbate in 0.9% saline) into the right ascending mesostriatal dopaminergic pathway at the following coordinates: 1) 2.5 I at anteroposterior (A/P)=−4.0, mediolateral (M/L)=−1.2, dorsoventral (D/V)=−7.8, toothbar=−2.4; and 2) 3.0 μl of 6-OHDA at A/P=−4.0; M/L=−0.8; DV=−8.0; toothbar=+3.4. The rate of injection was 1 μl/min with the cannula being left in place for 5 min before being slowly retracted. Animals were allowed to recover for two weeks in the animal care facility before given an amphetamine challenge (5.0 mg/kg ip), and their rotational scores were collected over a 70 min period using a computerized video activity monitor programmed for rotational behavior (Videomex®, Columbus Instruments, Columbus, Ohio). Only animals exhibiting a mean ipsilateral rotational score of eight or more complete full body turns per minute were included in the implant study.

Sixteen animals received double grafts of hNT neurons, seven received DA neurons, and 4 received LiCl-pre-treated DA-neurons. Three types of neurons were obtained from Layton Bioscience, Inc.: hNT neurons (6 wk RetA); DA neurons (4 wk RetA), and LiCl-pretreated DA-neurons—all stored at −180° C. until the time of transplantation. Two weeks following 6-OHDA lesions, rats were chosen for transplantation if they had a mean rotational score of 8 full body turns per minute. Beginning on the day of surgery, each animal received 10 mg/kg of cyclosporine A ip for the duration of the experiment. Prior to transplantation, the neurons were quickly thawed by placing the frozen vials in a water bath at 37° C. The neurons were then washed three time in DMEM /0.05% DNase (Sigma Chemical). The cells were suspended and the cell viability and suspension concentration calculated. The trypan blue dye exclusion method, which stains dead cells blue and fails to stain live cells, was used to assess cell viability.

The cell suspensions were stereotactically injected both intrastriatally and intranigrally using a technique previously described (Mendez and Hong, Brain Res 778: 194-205, 1997; and Mendez et al., 1996, ibid.). A specially designed capillary tip micropipette with an outer opening diameter of 50-70 μm is attached to a 2 μl Hamilton syringe and used to stereotactically implant the desired number of cells at a rate of 100 nl/min into both the SN and striatum (400,000 cells/site). Each animal received a total of about 800,000 cells. Injection of the cells into the dorsolateral striatum occurs at the following coordinates: 1) A/P =−1.3, M/L=−2.1, D/V=−5.5 and −4.3; 2) A/P=+0.6, M/L =−2.9; D/V=5.5 and −4.3; and 3) A/P=+0.3, M/L=−3.7, D/V=−5.5 and −4.3; toothbar=−3.3; coordinates from bregma and dorsal surface of the skull and the SN at the following coordinates: 1)A/P=−4.8, M/L=−2.0 D/V=−8.3 and −8.1; 2)A/P=−5.0, M/L=−2.3, D/V=−8.2 and −8.0; and 3) A/P=−5.3, M/L=−2.6, D/V=−8.1 and −7.9; toothbar=−3.3; coordinates from bregma and the dorsal surface of the skull.

At 3- and 6-wk post-transplantation, the rats were tested for rotational behavior. Comparison data are shown in FIG. 16 for post-lesion and 6 wk post-transplantation. The mean standard deviation (SD) rotations per minute with amphetamine challenge as described above. Data for hNT neurons are shown in the white bars, for DA neurons in the gray bars, and LiCl-pre-treated DA neurons in the black bars. There was no change for the LBS-neuron-treated rats, as expected. Although a reduction of rotational behavior was observed in DA-neuron and LiCl pre-treated DA-neuron groups, this reduction was not statistically significant.

At about 6 wk post-transplantation, the rats were euthanized with an overdose of anesthetic (supra) and perfused transcardially with 100 ml of 0.1 M phosphate buffer (PB), followed by 250 ml of 4% paraformaldehyde in 0.1M PB for 10 min. The brains were then removed from the cranium and fixed with 4% paraformaldehyde in 0.1M PB overnight before being stored for 24 hr in PB saline (PBS) containing 30% sucrose. With the freezing microtome, 40 μm coronal sections were made and stored in Millonig's solution (6% sodium azide in 0.1 M BP) until immunohistochemical processing of the sections could be performed. Following processing, sections were mounted in 0.1 M PB on gelatin-coated slides and coverslipped with Permount® mounting medium (Fisher Scientific).

Staining for the presence of tyrosine hydroxylase (TH) was performed using the primary rabbit anti-TH antibody (Ab; 1:2500; Pel Freeze Biologicals, Rogers, Ark.) and the ABC-kit (Vector Laboratories Canada, Inc., Burlington, Ontario, Canada). For this procedure the sections were pre-washed for 10 min in a solution of 10% methanol and 3% hydrogen peroxide and blocked in PB containing 0.3% Triton X-100 and 5% NSS for 1 hr. The sections were removed and incubated in a 1:2500 solution of rabbit polyclonal anti-TH Ab for 16 hrs. To visualize Ab binding, 1:500 biotinylated swine anti-rabbit IgG Ab (DAKO Diagnostics Canada, Inc., Mississauga, Ontario, Canada) was used, followed by a streptavidin-biotinylated HRP complex kit. The peroxidase activity was visualized by the addition of 3,3-diaminobenzidine (DAB). The sections were then washed in 0.1 M PB before mounting.

Staining for the presence of neural cell adhesion molecule (N-CAM)) was performed using the primary mouse anti-human N-CAM monoclonal antibody (Moc1, diluted 1 :1000, DAKO Diagnostics Canada, Inc.) and the ABC-kit. Briefly, the sections were pre-washed for 30 min in a solution of 10% methanol and 3% hydrogen peroxide and blocked in PB containing 0.3% Triton X-100 and 5% normal horse serum (NHS) for 1 hr. The sections were removed and incubated in a 1:1000 solution of monoclonal mouse anti-N-CAM (Moc 1) Ab for 16 hr. To visualize Ab binding, 1:250 biotinylated horse anti-mouse IgG Ab (Vector Laboratories, Inc., Burlington, Ontario, Canada) was used, followed by a streptavidin-biotinylated HRP complex kit. The peroxidase was visualized by the addition of DAB.

Staining for the presence of human neuron-specific enolase (NSE) was performed using the primary mouse anti-NSE monoclonal antibody (1: 100; Vector Laboratories Canada, Inc.) and the ABC-kit. The sections were pre-washed for 30 min in a solution of 10% methanol and 3% hydrogen peroxide and blocked in PB containing 0.3% Triton X-100 and 5% NHS for 1 hr. The sections were removed and incubated in a 1:100 solution of mouse monoclonal anti-hNSE Ab for 16 hr. To visualize Ab binding, 1:200 biotinylated horse anti-mouse IgG Ab was used, followed by a streptavidin-biotinylated HRP complex kit and DAB.

All animals that received both intrastriatal and intranigral hNT neuronal grafts had surviving grafts that were strongly immunostained for the presence of both human NSE and human NCAM. Analysis of the transplants by anti-NCAM immunohistochemistry revealed a strong staining of the entire graft area. Darkly NCAM stained cell-like structures could be seen within the graft boundary, and NCAM+ fibers extended beyond the graft-host interface in many of the animals. NSE staining produced a similarly intense pattern, with what appeared to be more darkly stained cells within the graft. NSE+ fibers extended beyond the graft-host interface at the level of the striatum; and in some cases, fibers extended greater than 100 μm into the surrounding host tissue.

Analyses for TH expression are summarized in FIG. 17. No TH+ cells were seen in either the striatum or the SN in animals with hNT neuron grafts (n=16). In 43% of animals with DA neuron grafts (n=4), TH+ cells were readily identified in both the striatum and SN. TH+ neurons were healthy, and their processes extended for variable distances in the host brain. After DA-neuron implants, there were 435.12±323.3 TH+ cells within the striatum and 393.68±204.70 TH+ cells within the SN. In 100% of animals receiving LiCl pre-treated DA-neurons, TH+ cells were observed in the intrastriatal and intranigral grafts. The mean number of TH+ cells within the intrastriatal and intranigral grafts was 489.39±18.09 and 319.68±142.08, respectively. There was no significant difference in the number of TH+ neurons between the DA-neuronal and the LiCl pre-treated DA-neuronal grafts (p>0.05). There was no significant difference in the number of TH+ cells between the intrastriatal and intranigral graft locations (p>0.05).

The lack of difference between surviving TH+ neurons in the striatum or SN suggests that the homotopic site (SN) does not influence the phenotype of hNT neurons. This contrasts with a report that hNT neurons differentiated to a dopaminergic phenotype under the influence of mouse caudatoputamen (Miyazono et al. J Comp Neurol 376:603-613, 1996). The lack of significant functional recovery most likely relates to the low number of TH+ neurons, as it has previously been demonstrated that the number of surviving TH+ neurons and fiber outgrowth strongly correlates with the extent of functional recovery. The present study shows that DA-neurons and LiCl-treated DA-neurons survive transplantation in the striatum and SN, integrate into the host, and express TH.

### Example 13

### Production and Testing of Minimally Induced (MI) Dopaminergic Neurons

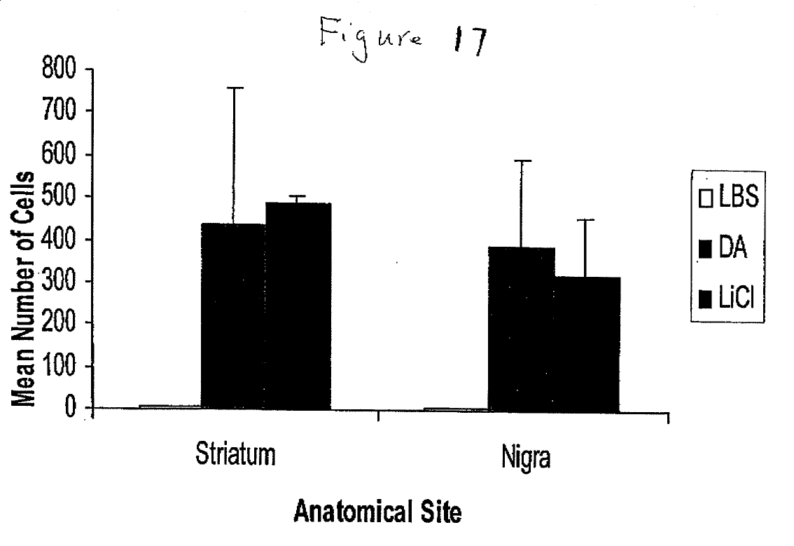

We examined RetA induction times less than 3 weeks and determined the minimum exposure that is need for neuronal differentiation with a pulse-chase Ret A experiment (FIG. 18). The cultures were induced with Ret A for 1, 2, 3, 4 or 5 weeks, but then switched to D/F-10 growth media for the remainder of the 5-week period. Next, all the cultures were replated after five weeks and treated with inhibitor for one week. Then the neurons were harvested and cryopreserved. The properties of these resulting neurons were compared, and the expressions of a variety of neuronal markers were analyzed by Western analysis and immunohistochemistry.

Neuron production was determined for 1 through 5 weeks of RetA induction. The results are shown in Table 4. As can be seen, the highest yield of neurons was obtained after three weeks of exposure to RetA with a total yield of 7.4×10⁷ cells per flask processed. It is interesting to note that with as little as I week of exposure to RetA, the NT-2 precursor cell committed to differentiate into neurons. However, the yield of neurons from this 1W RetA was very low, and these neuronal cells did not exhibit the desired morphology and adhesion properties. Isolation of purified neuronal cells was very difficult from the 1W and 2W batches, which interfered with further analysis of these samples.

TABLE 4
Cell and Neuronal Cell Yield from RetA Pulse Chase cultures.

| | Cell yield after | Neuron yield per | |
|---|---|---|---|
| Weeks in | RetA treatment per | RetA T-175 flask | Percent viability |
| RetA | T-175 flask (× 10⁶) | (× 10⁶) | of bulk neurons |
| 1 | 292 | 7.8 | 80% |
| 2 | 611 | 50.3 | 81% |
| 3 | 573 | 74 | 80% |
| 4 | 401 | 59.2 | 81% |
| 5 | 335 | 57.4 | 85% |

The TH expression was easily detected by Western Blot as early as after 2W RetA and increased to its highest level in the 3W pulse-chased cultures. There appears to be slightly less TH in 4W RetA exposed cultures, but by 5W there is a dramatic decrease in TH-expressing cells, as well as the intensity of TH staining of neuronal cells. Other neuronal markers showed a different trend. BCl-2 reached maximum expression after 5W of RetA, as determined both by immunocytochemistry and Western analysis. Muscarinic receptor was not detected until 3W and demonstrated a more subtle level of expression, but the percentage of positive cells continued to increase until 5W. Still other neuronal markers remained high and unchanged when analyzed by immunofluorescence, including the neurotransmitter enzyme acetylcholine esterase (MAB304), NCAM (MOC 1), Axonal Neurofilament-P+++ (HO-14), Neurofilament-M (RMO-254) and MAP2b (T-34). A summary of the immunostaining profile of the cells is shown in Table 5.

TABLE 5
Comparison of Immunostaining reactivity
of RetA Pulse Chase Neuronal cells

| Antibody | | | | |
|---|---|---|---|---|
| Marker | Week 2 | Week 3 | Week 4 | Week 5 |
| TH | ++ | +++ | +++ | ++ |
| BCl-2 | ++ | +++ | +++ | ++++ |
| T-34 | ++++ | ++++ | ++++ | ++++ |
| RMO-254 | +++++ | +++++ | +++++ | +++++ |
| MOCI | +++ | +++ | +++ | +++ |
| MuscarinicM2 | +/− | + | + | +++ |
| MAB 304 | +++ | +++ | +++ | +++ |

Cells were plated in pretreated lysine/laminin 4 well slides at 100,000 cells per well for 2 days at 37° C. Slides were processed as described above. Symbols represent the percentage and intensity of neuronal cells that immunostained for the different antibodies in comparison to HO 14.4 immunoreactivity, which routinely stains++. On this scale, +=weak and few cells immunoreacted; ++=>25-30% of cells immunoreacted; +++=>50% of cells immunoreacted with moderate to strong intensity; ++++=all cells immunoreacted with very intense fluorescence.

### Comparison of DA vs MI vs hNT Cells

DA Neurons were compared with hNT neurons above. Parallel inductions were set up to produce hNT (5W), DA (4W) and MI neurons (3W +pulse chase), and neuronal cells were purified and analyzed for expression as a direct comparison. Notably 3W + pulse-chase time course produced a high yield of neuronal cells. The level of TH expression in MI cells was significantly higher than in the hNT-neurons, even though both cell types were cultured for the same total time of 5 weeks (FIG. 18). We also compared the cell yield, % viability and TH expression both from bulk harvests of hNT, DA and MI cultures including the co-culturing of these cells on rat cortex and striatal astrocytes to analyze the maturation of these neurons in vitro. These alternative 3W, 4W, and 5W cells were also cryopreserved; and their viability upon thaw, neurite outgrowth activity, apoptotic index, and stability of TH expression upon recovery was determined.

Cell yields were significantly higher from the MI cultures than the DA cultures. No significant difference was seen in parallel cultures in % viability upon harvest or after freezing of the cells. MI cells have higher levels of TH in Western analysis (lane 3 of FIG. 19) and have significantly more TH-expressing cells by immunofluorescence. Upon recovery from freezing, the MI and DA cells continued to express high levels of TH (lanes 7 and 8). Co-culturing of the thawed DA and MI cells for 1 or 2 weeks on rat cortex astrocytes showed that the MI neurons maintained stable levels of TH-expressing cells even after 2W in culture; whereas, the TH level decreased in DA cells.

Post-Cryopreservation Survival and Apoptosis

MI, DA and hNT neurons as well as NT2 precursors were stored at −180 ° C. prior to use. Freshly thawed cells resuspended in the medium containing DMEM (Gibco, BRL, Grand Island, N.Y.) and 10% fetal bovine serum (Gibco, BRL) were plated on poly-L-lysine coated eight-well chamber slides at a concentration of 100,000 cells/cm². After 24 h the plating media was switched to DMEM:F12 (Gibco, BRL) containing 0.1% ITS (Sigma), and Gentamicin (50,μg/ml, Sigma). Cultures were either maintained in the second medium for 1 day (1 DIV) or for 5 days (5 DIV), then rinsed in 0.1 M phosphate buffered saline (PBS) and fixed with 4% paraformaldehyde.

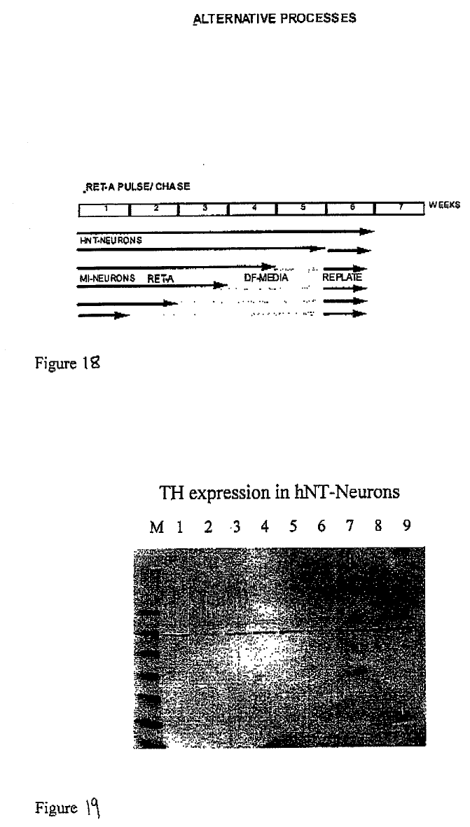

Survival and the morphological appearance of living, non-fixed cultures neurons and precursors were assessed from slides stained with fluorescein diacetate (FDA)-propidium iodide (PI) at DIV1 or 5. Only those platings, which revealed the vast majority (85-95%) of healthy FDA+ cells (FIG. 20) evenly distributed throughout a culture dish were selected and processed for apoptosis or TH/DAPI-immunocytochemistry (see below). FIG. 20 shows assessment of cell viability FIGS. 20A and 20B show NT2 cells, and FIGS. 20C and 20D DA cells (previously treated with RA for 4 w). Cells cultured for 1 day are shown in FIGS. 20A and 20C, and cells cultured for 5 days are shown in FIGS. 20B and 20D. The vast majority of cells were labeled by FDA, which fluoresced green and was taken up by living cells; the PI fluoresced red and passively accumulated in dead cells (arrow). The bar represents 100 μm.

For the histological determination of apoptosis in vitro, the NeuroTACS™ In Situ Apoptosis Detection Kit (R&D systems) was used to identify apoptotic nuclei. Freshly fixed cultures were first permeabilized with NeuroPore reagent, and endogenous activity was quenched using H₂O₂. DNA fragmentation in individual apoptotic cells was visualized by detection of biotinylated nucleotides incorporated into the free 3′-hydroxyl residues of these DNA fragments. A streptavidin-conjugated horseradish peroxidase bound to the biotinylated DNA fragments generated brown precipitates in the presence of diaminobenzidine (DAB). Blue counterstaining was used for easier identification of cells. The positive controls were generated by brief treatment of cells with nuclease prior to labeling in order to generate DNA strand breaks in virtually all cells. Negative controls consisted of slides in which terminal deoxynucleotidyl transferase (tdt) was omitted from the reaction mixture. The number of apoptotic nuclei versus total number of cells was determined from three independent culture platings for every RA exposure and time point (1 and 5 DIV). The number of apoptotic and non-apoptotic cells was counted using a 20×objective placed over two randomly selected non-overlapping sites per well (4 wells/plating, 3 platings in total). Percentages were determined as a ratio of apoptotic cells/total number of cells multiplied by 100. The mean values + SEM from 0, 3, 4 and 5 weeks RA exposures at each time point were compared using a one-way analysis of variance followed by Dunnet's post hoc comparisons.

NT2 precursors at 1 DIV plated at the same density as induced cells covered uniformly the surface of the culture dish. NT2 cell bodies were large and flat, occasionally sending out short processes. Numerous NT2 cells were in various stages of mitosis, easily distinguishable after blue counterstaining. The number of apoptotic positive nuclei was very low (3.6%). In 5 DIV cultures, the proliferating precursor cells completely covered the surface of the well forming a tightly packed carpet-like monolayer. Even after longer survival, NT2 precursors revealed low levels of tdt labeling (4.4%). Positive nuclei were usually round with distinct fragmentation (Table 6).

TABLE 6
Apoptosis in NT2, MI (3W), DA (4W) and hNT (5W) Cells

Percentage of tdt-labeled cells

| DIV | NT2 | 3wRA | 4wRA | 5wRA |
|---|---|---|---|---|
| 1 | 3.6 ± 0.1 | 10.9 ± 0.5 | 12.5 ± 2.0 | 12.5 ± 0.5 |
| | (2768) | (1292)* | (3562)* | (2310)* |
| 5 | 4.4 ± 0.3 | 15.8 ± 1.5 | 15.4 ± 1.3 | 15.3 ± 1.0 |
| | (1728) | (3763)* | (3572)* | (3284)* |

Data represent the percentage of apoptotic cells vs total number of cells (numbers in parentheses) ±SEM. * Significant difference between non-RA and RA-treated cultures in both studied time points; p <0.01.

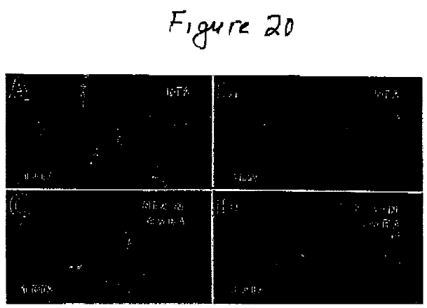

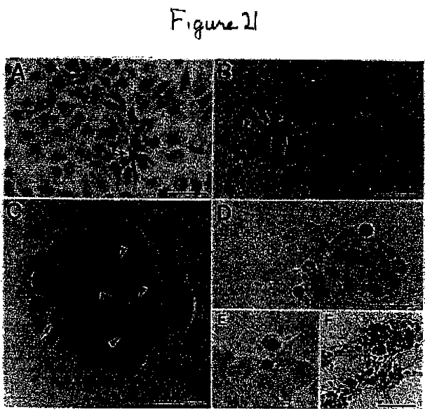

After exposure to RA, the morphological appearance of differentiated neurons was substantially changed. After 1DIV individual cells or clusters containing neurons with small usually round bodies and only few, short processes were found. When compared to NT2 undifferentiated cultures, significantly higher numbers of apoptotic-positive nuclei were detected in every RA-treated group (Table 6). The appearance of DNA condensation varied from small round darkly stained nuclei to those showing a darkly labeled nuclear periphery and weakly labeled center (also called “halo” morphology) or fragmented nuclei broken into several intensely tdt-positive pieces (FIG. 21). FIGS. 21A-21F show apoptosis in NT2 and induced cells cultured for 5 days. FIG. 21A is a bright-field photomicrograph showing apoptotic nuclei (dark brown, e.g., arrows) in cultured NT2 cells. The bar represents 50 μm. FIG. 21B shows a group of DA neurons (previously treated with RetA for 4 w) with several apoptotic nuclei (arrows). The blue counterstain was used to visualize the cell bodies. The bar represents 50 μm. FIG. 21C shows a clump of DA neurons (4W RetA) showing nuclei with a single (arrowheads) or multiple lobes of condensed chromatin (arrow). The bar represents 25 μm. FIG. 21D is another example from the group of MI cells exposed previously to 3W RetA-treatment. Apoptotic cells indicated by arrows have “halo” morphology. The bar represents 50 μm. FIG. 21E shows that in some dying MI neurons (arrow), cytoplasm is still visible/present. This bar represents 50 μm. FIG. 21F shows the positive control (see above) from the same experimental group as in FIG. 21B. Bar represents 100 μm. Overall the most frequently seen was the first group, with dark compact nuclear staining. In many instances, the cytoplasm of dying cells was shrunken or substantially reduced.

After 5 DIV induced cultures contained numerous clusters of well developed cells with typical neuronal morphology. Numerous cells extended long and branching processes interconnecting adjacent cell groups. Apoptotic nuclei with variable morphology were observed across all RetA-treated groups.

Nuclear fragmentation in the previously frozen cells was revealed with a polyclonal tyrosine hydroxylase (TH) antibody (Pel Freez, diluted 1:500 in phosphate-buffered saline (PBS) containing 0.05% Triton X-100. For immunofluorescence TH was visualized using rhodamine conjugated to goat anti-rabbit IgG (Alexa Molecular Probes Inc., Eugene, Oreg.). For nuclear staining the same cultures were exposed for 10 min to the intercalating dye bisbenzimide (Hoechst 33258, Sigma) at 2 μg/ml, washed twice with PBS and observed with standard epi-illumination fluorescence microscopy (Olympus BX60) equipped with appropriate rhodamine and DAPI filters.

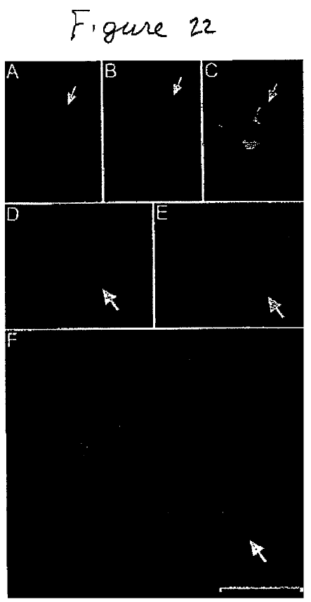

After 1 or 5 days in culture, TH-immunoreactivity was not detected in NT2 precursors. In all RA-treated groups, there were healthy TH+cells with non-apoptotic nuclei. However, careful inspection of cultures revealed the presence of a subpopulation of TH+ cells with decreased TH-immunoreactivity and reduced size and which contained either condensed or fragmented nuclear chromatin (FIGS. 22). FIGS. 22A-22F display the results of TH/DAPI immunocytochemistry. FIGS. 22A-C are fluorescent photomicrographs showing TH+ cells (A, red, arrow), Hoechst+ nuclei (B, blue, arrowhead) and double-labeled (C, TH+/Hoechst+, double-arrow) MI neurons (treated with RetA for 3W) cultured for 1DIV. Most TH+ neurons had healthy, non-apoptotic nuclei. The bar represents 65 μm in A-C. FIGS. 22D-F are also fluorescent photomicrographs showing a clump of hNT cells cultured for 1DIV (previously subjected to RA treatment for 5 w). TH+neuron indicated by arrow in FIG. 22D reveals fragmented nucleus (arrow), shown in FIGS. 22E and F at higher magnification. The bar represents 27 μm in FIGS. 22D-F. The diminished intensity of TH immunolabeling in dying neurons, compared to healthy TH+ cells in the same culture, may indicate the loss of the neurotransmitter phenotype. However, the percentage of dying TH+ cells at both time points (1 and 5 DIV) was relatively low and did not affect the entire dopaminergic population (Table 7).

TABLE 7
Apoptosis in NT2, MI (3W), DA (4W) and hNT (5W) Cells

| | | Percentage of apoptotic+/TH+ cells | | |
|---|---|---|---|---|
| DIV | NT2 | 3W RetA | 4W RetA | 5W RetA |
| 1 | 0 | 17.1 (99) | 14.9 (194) | 16.3 (184) |
| 5 | 0 | 23.5 (136) | 20.1 (164) | 19.2 (197) |

Data represent the percentage of dying TH+ induced neurons vs total number of TH+ induced neurons (numbers in parentheses) in 10 non-overlapping microscopic fields per plating (3 platings/condition) observed at 40X magnification. Hoechst 33258 intercalating dye was used to detect apoptotic nuclei with condensed chromatin.

Because cell death in cultured DA and MI neurons occurred less frequently than in primary mixed cultures derived from embryonic rat VM, the DA and MI neurons are more likely to successfully survive transplant than have been VM cells.

### Example 14

### MPTP non-human model of PD

A non-human higher mammal study (e.g., in primates) bridges the gap between rodent and human. This study demonstrates efficacy, long-term effects, scale-up to doses close to humans, general and specific safety, and valid neurosurgical procedures in preparation for clinical trials for the indication of PD. Non-human primates may be unilaterally lesioned in the caudate and putamen with 1-methyl-4-phenyl-1,2,3,6-tetrahydropyridine (MPTP) to produce hemiparkinsonian-like symptoms that provide a good model for evaluation of interventions in PD (19). Following the lesion, animals spontaneously circle in the direction of the lesion, but in response to apomorphine, they reverse and circle contralateral to the side of the lesion. Associated motor and behavioral symptoms include generalized slowing, rigidity and tremors characteristic of PD. After the lesions stabilize, optimized DA/hNT neurons are administered to at least one part of the lesioned area. Possible administration protocols call for delivery of the neurons in 1-20 tracks, preferably 1-10 tracks, more preferably 1-6 tracks and most preferably about 4 tracks. It is understood that one or more doses of cells is administered in each track, once as the needle is initially placed and optionally again or repeatedly as the needle is partially withdrawn. The amounts of cells to be delivered in each track varies from about 5×10⁴ to 5×10⁶ cells per dose, preferably 1×10⁵ to 2×10⁶ cells per dose, most preferably about 5×10⁵ to 1.5×10⁶ per dose, and most preferably 10⁶ per dose; however, these dose ranges are to be modified based on the identification of the optimal dose in rodents and the appropriate scale-up factor for higher mammals. The cells or appropriate control(s) are administered to caudate and putamen of MPTP-lesioned animals. Animals undergo repeated behavioral testing at suitable intervals before being sacrificed for histological analyses. Histologic examination will be done for tumor formation, brain infection, and graft rejection.

The foregoing description and examples are intended only to illustrate, not limit, the disclosed invention.

1. A method of producing dopaminergic neuronal cells suitable for transplantation in dopamine deficiencies, said transplantable neuronal cells being derived from progenitor cells,
a. providing progenitor cells which lack at least one indicator of neuronal cell differentiation;
b. treating the progenitor cells with an inducing agent for a time period sufficient to optimize expression of tyrosine hydroxylase and to induce the presence of at least one indicator of neuronal cell differentiation to produce a plurality of dopaminergic, differentiated neuronal cells; and
c. minimally replating with an inhibitor to optimize the dopaminergic phenotype and a purified harvest; and.
d. harvesting the dopaminergic, differentiated neuronal cells.

2. The method of claim 1, wherein the step of providing progenitor cells provides mammalian cells.

3. The method of claim 1, wherein the step of providing progenitor cells provides human NT2/D1 cells.

4. The method of claim 1, wherein the step of providing progenitor cells provides mammalian fetal cells.

5. The method of claim 1, wherein the step of providing progenitor cells provides mammalian stem cells.

6. The method of claim 1, wherein step (c) also includes adding at least one lithium salt.

7. The method of claim 1, wherein step (c) is followed by an additional step of co-culturing with at least one cell type which stabilizes or improves the dopaminergic phenotype of the cells.

8. The method of claim 7, wherein the co-culturing step is co-culturing with human bone marrow stem cells, fetal stem cells, or Sertoli cells.

9. The method of claim 7 wherein the co-culturing step comprises co-culturing with Sertoli cells, human bone marrow stem cells, or a combination thereof.

10. The method of claim 1 wherein the step of treating the progenitor cells comprises applying retinoic acid or retinoids thereto.

11. A dopaminergic neuronal cell suitable for transplantation into an individual having a dopaminergic deficiency, said cell comprising
a post-mitotic differentiated neuronal cell which expresses tyrosine hydroxylase and at least one other indicator of neuronal cell differentiation, said cell having undergone induction from an undifferentiated cell.

12. A human post-mitotic dopaminergic cell suitable for transplantation into a human having a dopaminergic deficiency, said cell comprising a differentiated neuronal cell which expresses tyrosine hydroxylase and at least one other indicator of neuronal cell differentiation, said cell having undergone induction from an undifferentiated human cell.

13. A human dopaminergic cell suitable for transplantation into a human having a dopaminergic deficiency, the cell comprising a differentiated human neuronal cell that expresses tyrosine hydroxylase and bcl-2, said cell being capable of synthesizing dopamine and having improved survival after transplantation.

14. A method of improving the survival of human neuronal cells for transplantation, said method comprising the steps of
a. providing a culture of human cells;
b. adding a lithium salt to the human cell culture for a sufficient time to enhance expression of bcl-2;
c. testing cells from the treated cell culture for the presence of bcl-2; and
d. isolating the cells from the culture to produce an isolated cell preparation; and
e. testing the isolated cell preparation for sterility before packaging the cells for transport.

15. A pharmaceutical dosage form of human non-fetal cells suitable for transplantation in Parkinson's Disease comprising
isolated, purified, neuronal cells, the neuronal cells expressing tyrosine hydroxylase, D2 dopamine receptor, and aldehyde dehydrogenase-2; and
a pharmaceutical diluent.

16. The transplantable neuronal cells of claim 13, wherein the lithium salt is lithium chloride.

17. The method of claim 15, wherein the lithium salt is lithium chloride.

18. A chimeric non-human mammal wherein the mammal comprises post-mitotic dopaminergic neuronal cells implanted in the brain of the mammal.

19. A method of preparing human neuronal cells suitable for treating Parkinson's Disease, the method comprising:
a) providing NT2/D1 cells;
b) culturing NT2/D1 cells with an inducing agent for a time sufficient to optimize TH expression therein;
c) replating and culturing the TH-optimized cells in mitotic inhibitor; and
d) separating the TH-optimized neuronal cells from the replate culture.

20. The method of claim 19, additionally comprising the steps of
e) replating the TH-optimized cells on a confluent feeder cell layer, the cell layer being chosen from cells which stabilized TH production, including bone marrow stem cells, TM4 Sertoli cells, glioma cells, or a combination thereof; and
f) isolating the TH-optimized and stabilized cells from the replate medium.

21. A pharmaceutical composition for treating Parkinson's Disease, the composition comprising
isolated, purified, neuronal cells, the neuronal cells expressing tyrosine hydroxylase, D2 dopamine receptor, and aldehyde dehydrogenase-2;
cells capable of stabilizing tyrosine hydroxylase production; and
a pharmaceutical diluent.

22. The composition of claim 21 in which the stabilizing cells are Sertoli cells, bone marrow stem cells or a combination thereof.

23. A purified human dopaminergic cell type, the cells having been cultured from NT2 cells, treated for about two to three weeks with an inducing agent, cultured for about two weeks with growth media without an inducing agent or mitotic inhibitor, cultured for about one week with at least one mitotic inhibitor, harvested and placed in a diluent.

24. A method of producing neurotransmitter phenotype cells, selected from the group of dopaminergic, serintinergic, cholinergic, and gabanergic cells, suitable for transplantation in neurodegenerative deficiencies or abnormal neurological conditions, said transplantable neurotransmitter phenotype cells being derived from progenitor cells,
a. providing progenitor cells which lack at least one indicator of neuronal cell differentiation;
b. treating the progenitor cells with an inducing agent for a time period sufficient to optimize expression of a specific neurotransmitter marker and to induce the presence of at least one indicator of neuronal cell differentiation to produce a plurality of desired neuronal cells; and
c. minimally replating with an inhibitor to optimize the desired phenotype and a purified harvest; and.
d. harvesting the desired neuronal cells.

25. A neurotransmitter phenotype cell selected from the group of dopaminergic, serintinergic, cholinergic, and gabanergic cells, suitable for transplantation into an individual having a neurodegenerative deficiency, said cell comprising
a post-mitotic differentiated neuronal cell which expresses a specific neurotransmitter marker and at least one other indicator of neuronal cell differentiation, said cells having undergone induction from an undifferentiated cell.

26. A human post-mitotic neurotransmitter cell suitable for transplantation into a human having a neurodegenerative deficiency, said cell comprising a differentiated neuronal cell which expresses a specific neurotransmitter marker and at least one other indicator of neuronal cell differentiation, said cell having undergone induction from an undifferentiated human cell.

27. A chimeric non-human mammal wherein the mammal comprises post-mitotic neurotransmitter phenotype neuronal cells, selected from the group of dopaminergic, serintinergic, cholinergic, and gabanergic cells, implanted in the brain of the mammal.
